(19) United States
(12) Patent Application Publication
(10) Pub. No.: US 2025/0091983 A1
(43) Pub. Date: Mar. 20, 2025

(54) CYCLOPENTANE AND CYCLOHEXANE VARIANTS OF 6-PHENYLHEXANAMIDE MITOFUSIN ACTIVATORS AND METHODS FOR USE THEREOF

(71) Applicant: MITOCHONDRIA EMOTION, INC., Hamilton, OH (US)

(72) Inventor: Gerald W. DORN, II, Sullivan's Island, SC (US)

(73) Assignee: MITOCHONDRIA EMOTION, INC., Cambridge, MA (US)

(21) Appl. No.: 18/294,389

(22) PCT Filed: Aug. 3, 2022
(86) PCT No.: PCT/US2022/039326

§ 371 Date: Feb. 1, 2024

Related U.S. Application Data
(60) Provisional application No. 63/228,665, filed on Aug. 3, 2021.

(51) Int. Cl. C07C 233/60; A61K 31/165

(52) U.S. Cl. CPC C07C 233/60; A61K 31/165; C07C 2601/08; C07C 2601/14

(57) ABSTRACT

The present disclosure relates to compounds of Formula (I) or pharmaceutically acceptable salts thereof. The present disclosure also relates to uses of the compounds, e.g., in treating or preventing diseases, disorders, or conditions (e.g., associated with mitochondria).

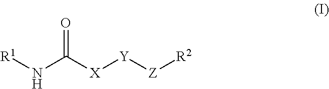

### RELATED APPLICATION

The present application claims benefit of U.S. Provisional Application No. 63/228,665, filed Aug. 3, 2021, which is hereby incorporated by reference in its entirety.

### BACKGROUND

Mitochondrial dysfunction may contribute to various types of neurodegenerative diseases. Defective mitochondrial fusion or fission may be especially problematic in this regard, especially when imbalanced fusion and fission lead to mitochondrial fragmentation. Among the many neurodegenerative diseases in which mitochondrial dysfunction has been implicated include, for example, Charcot-Marie-Tooth disease, amyotrophic lateral sclerosis (ALS), and Huntington's disease.

Mitochondrial fusion is initiated by outer mitochondrial membrane-embedded mitofusin (MFN) proteins whose extra-organelle domains extend across cytosolic space to interact with counterparts on neighboring mitochondria. The physically linked organelles create oligomers of varying sizes. Mitofusins subsequently induce outer mitochondrial membrane fusion mediated by catalytic GTPase. Aberrant mitofusin activity is believed to be a primary contributor to mitochondrial-based neurodegenerative diseases. For these reasons, mitofusins are attractive targets for drug discovery.

There remains a need for new compounds that target mitofusins. The present disclosure addresses the need.

### SUMMARY

In some aspects, the present disclosure provides a compound to Formula (I):

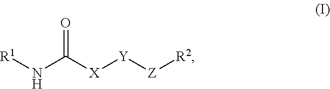

or a pharmaceutically acceptable salt thereof, wherein
R¹ is cycloalkyl or heterocycloalkyl, wherein the cycloalkyl or heterocycloalkyl is optionally substituted with one or more
each R^{X} independently is halogen, cyano, azo, —OR^{X1}, C₁-C₁₀ alkyl-OH, C₁-C₁₀ alkyl-NH₂, —N(R^{X1})₂, C(O)—R^{X}, C(O)—OR^{X1}, —OC(O)R^{X1}, C₁-C₁₀ alkyl-C(O)—N(R^{X1})₂, C₁-C₁₀ alkyl-N(R^{X1})C(O)R^{X1}, C(O)—N(R^{X1})₂, —N(R^{X1})C(O)R^{X1}, S(R^{X1}), S(O)(R^{X1}), S(O)₂(R^{X1}), S(O)₂—N(R^{X1})₂ oxo, C₁-C₁₀ alkyl, C₃₋₈ cycloalkyl, 3- to 10-membered heteroaryl, 3- to 10-membered heterocyclyl, C₆-C₁₀ aryl, wherein the alkyl, cycloalkyl;
R² is aryl or heteroaryl, wherein the aryl or heteroaryl optionally substituted with one or more R;
each R^{Y} independently is halogen, cyano, —OR^{Y1}, —N(R)^{Y1})₂, C₁-C₁₀ alkyl, or C₃-C₁₀ cycloalkyl;
each R^{X1} and R^{Y1} independently is H, C₁-C₆ alkyl, C₆-C₁₀ aryl, halogen, —C(O)NH₂, —OH, —NH₂, cyano, C₃-C₈ cycloalkyl, 3- to 10-membered heteroalkyl, 3- to 10-membered heterocyclyl, C(O)—R^{X2}, C(O)—OR^{X2}, S(O)(R^{X2}), S(O)₂(R^{X2}), azo, thiophene, or indole;
R^{X2} independently is H or C₁-C₆ alkyl;
X is (CH₂)ₐ;
Y is cyclopentyl or cyclohexyl;
Z is (CH₂)_{b};
a is 0 or 1; and
b is 1, 2, or 3.

In some aspects, the present disclosure provides a compound to Formula (I):

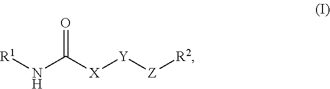

or a pharmaceutically acceptable salt thereof, wherein
R¹ is C₃-C₁₀ cycloalkyl or 3- to 10-membered heterocycloalkyl, wherein the cycloalkyl or heterocycloalkyl is optionally substituted with one or more R^{X};
each R^{X} independently is halogen, cyano, —OR^{X1}, —N(R^{X1})₂, oxo, C₁-C₁₀ alkyl, or C₃-C₁₀ cycloalkyl;
R² is phenyl optionally substituted with one or more R^{Y};
each R^{Y} independently is halogen, cyano, —OR^{Y1}, —N(R^{Y1})₂, C₁-C₁₀ alkyl, or C₃-C₁₀ cycloalkyl;
each R^{X1} and R^{Y1} independently is H or C₁-C₆ alkyl;
X is (CH₂)ₐ;
Y is cyclopentyl or cyclohexyl;
Z is (CH₂)_{b};
a is 0 or 1; and
b is 1, 2, or 3.

In some aspects, the present disclosure provides a method of preparing a compound described herein.

In some aspects, the present disclosure provides a pharmaceutical composition comprising any compound described herein and a pharmaceutically acceptable excipient.

In some aspects, the present disclosure provides a method of treating diseases, disorders, or conditions, comprising administering to a subject any compound described herein in a pharmaceutical composition.

In some aspects, the present disclosure provides any compound described herein in a pharmaceutical composition for use for treating diseases, disorders, or conditions, comprising administering to a subject.

In some aspects, the present disclosure provides use of any compound described herein in a pharmaceutical composition in the manufacture of a medicament for treating diseases, disorders, or conditions, comprising administering to a subject.

In some aspects, the present disclosure provides a method of activating mitofusin in a subject, comprising administering the compound or the pharmaceutical composition of any one of the preceding claims.

In some aspects, the present disclosure provides any compound described herein in a pharmaceutical composition for use in activating mitofusin in a subject.

In some aspects, the present disclosure provides use of the any compound described herein in a pharmaceutical composition in the manufacture of a medicament for activating mitofusin in a subject.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting. In the case of conflict between the chemical structures and names of the compounds disclosed herein, the chemical structures will control.

Other features and advantages of the disclosure will be apparent from the following detailed description and claims.

### DETAILED DESCRIPTION

Without wishing to be bound by theory, it is understood that the compounds disclosed herein may be effective in activating mitofusin. Thus, the compounds may be useful for treating various diseases and disorders, including mitochondria associated diseases, disorders, or conditions.

Various N-(cycloalkyl or heterocycloalkyl)-6-phenylhexanamide compounds may be potent mitofusin activators (U S. Patent Application Publication 2020/0345669). N-(trans-4-hydroxycyclohexyl)-6-phenylhexanamide (Compound 1) could be a particularly potent example of a mitofusin activator (U.S. Patent Application Publication 2020/0345668).

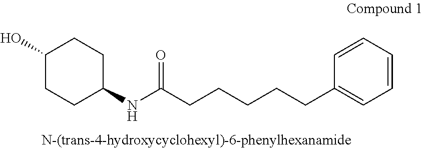 [SMILES: O=C(CCCCCc1ccccc1)N[C@H]1CC[C@H](O)CC1]

It was discovered that by introducing rigidity into the methylene chain extending between the amide carbonyl and the phenyl ring of Compound 1, the plasma half-life and neurological bioavailability, as evidenced by PAMPA values, may be surprisingly and significantly improved without significantly altering the EC₅₀ value of the parent compound. A particularly efficacious mitofusin activator may be obtained by fusing the two methylene groups adjacent to the amide carbonyl together as a cyclopropyl group (cyclopropane ring), the structure of which is shown in Compound 2.

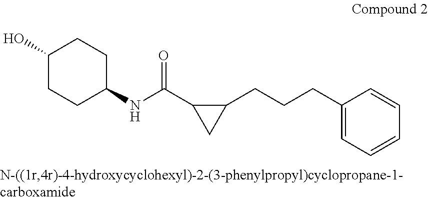 [SMILES: O=C(N[C@H]1CC[C@H](O)CC1)C1CC1CCCc1ccccc1]

The mitofusin activation capabilities (EC₅₀ values) and PAMPA values may remain at least comparable to those of the parent compound when the cyclopropyl group is located elsewhere in the methylene chain, as shown for the mitofusin activator having a structure represented by Compound 3 below.

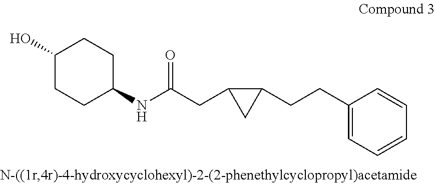 [SMILES: O=C(CC1CC1CCc1ccccc1)N[C@H]1CC[C@H](O)CC1]

The cyclobutyl variant of Compound 2, with the same number of atoms extending between the amide carbonyl and the phenyl ring (5 total bridging atoms) and having a structure represented by Compound 4 below, maintains the PAMPA value of the mitofusin activator having a structure represented by Formula 2 and with only a slight decrease in EC₅₀ value.

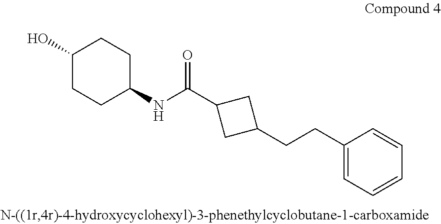 [SMILES: O=C(N[C@H]1CC[C@H](O)CC1)C1CC(CCc2ccccc2)C1]

In view of the fact that increasing the ring size from cyclopropyl in Compound 2 to cyclobutyl in Compound 4 does not significantly further increase the PAMPA value relative to that of the mitofusin activator having a structure represented by Compound 1, it was surprisingly discovered that further increasing the ring size to cyclopentyl or cyclohexyl may approximately double the PAMPA value compared that of Compound 2. Moreover, cyclopentyl and cyclohexyl variants may maintain EC₅₀ values similar to those of Compound 2. Accordingly, the mitofusin activators described herein may exhibit increased blood-brain permeability by virtue of their elevated PAMPA values while still maintaining high mitofusin activation capabilities.

### Compounds of the Present Disclosure

Any structural feature described herein (e.g., for any exemplary formula described herein) can be used in combination with any other structural feature(s) described for any exemplary formula described herein.

In some aspects, the present disclosure provides a compound to Formula (I):

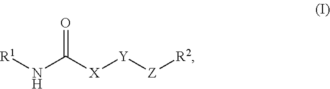

or a pharmaceutically acceptable salt thereof, wherein
R¹ is cycloalkyl or heterocycloalkyl, wherein the cycloalkyl or heterocycloalkyl is optionally substituted with one or more
each R^{X} independently is halogen, cyano, azo, —OR^{X1}, C₁-C₁₀ alkyl-OH, C₁-C₁₀ alkyl-NH₂, —N(R^{X1})₂, C(O)—R^{X1}, C(O)—O^{X1}, —OC(O)R^{X1}, C₁-C₁₀ alkyl-C(O)—N(R^{X1})₂, C₁-C₁₀ alkyl-N(R^{X1})C(O)R^{X1}, C(O)—N(R^{X1})₂, —N(R^{X1})C(O)R^{X1}, S(R^{X1}), S(O)(R^{X1}), S(O)₂(R^{X1}), S(O)₂—N(R^{X1})₂, oxo, C₁-C₁₀ alkyl, C₃₋₈cycloalkyl, 3- to 10-membered heteroaryl, 3- to 10-membered heterocyclyl, C₆-C₁₀ aryl, wherein the alkyl, cycloalkyl;
R² is aryl or heteroaryl, wherein the aryl or heteroaryl optionally substituted with one or more R^{Y};
each R^{Y} independently is halogen, cyano, —OR¹, —N(R^{Y})₂, C₁-C₁₀ alkyl, or C₃-C₁₀ cycloalkyl;
each R^{X1} and R^{Y1} independently is H, C₁-C₆ alkyl, C₆-C₁₀ aryl, halogen, —C(O)NH₂, —OH, —NH₂, cyano, C₃-C₈ cycloalkyl, 3- to 10-membered heteroalkyl, 3- to 10-membered heterocyclyl, C(O)—R^{X2}, C(O)—OR^{X2}, S(O)(R^{X2}), S(O)₂(R^{X2}), azo, thiophene, or indole;
R^{X2} independently is H or C₁-C₆ alkyl;
X is (CH₂)ₐ;
Y is cyclopentyl or cyclohexyl;
Z is (CH₂)_{b};
a is 0 or 1; and
b is 1, 2, or 3.

In some aspects, the present disclosure provides a compound of Formula (I):

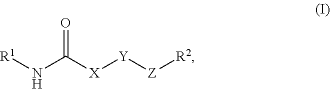

or a pharmaceutically acceptable salt thereof, wherein
R¹ is C₃-C₁₀ cycloalkyl or 3- to 10-membered heterocycloalkyl, wherein the cycloalkyl or heterocycloalkyl is optionally substituted with one or more R^{X};
each R^{X} independently is halogen, cyano, —OR^{X1}, —N(R^{X1})₂, oxo, C₁-C₁₀ alkyl, or C₃-C₁₀ cycloalkyl;
R² is phenyl optionally substituted with one or more R^{Y};
each R^{Y} independently is halogen, cyano, —OR^{Y1}, —N(R^{Y1})₂, C₁-C₁₀ alkyl, or C₃-C₁₀ cycloalkyl;
each R^{X1} and R^{Y1} independently is H or C₁-C₆ alkyl;
X is (CH₂)ₐ;
Y is cyclopentyl or cyclohexyl;
Z is (CH₂)_{b};
a is 0 or 1; and
b is 1, 2 or 3.

In some embodiments, X (where present), Y and Z collectively form a 5-atom bridge between the amide carbonyl and R².

In some embodiments, X, Y, and Z collectively form a 6-atom bridge between the amide carbonyl and R².

In some embodiments, the 5- or 6-atom bridge formed by X, Y, and Z refers to the shortest continuous chain of atoms extending between the amide carbonyl and the phenyl ring. For example, in Formula 6, a is 0, Y is 1,3-cyclopentyl, b is 2, the bridge has 5 atoms.

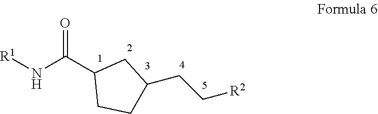

In some embodiments, R¹ is unsubstituted C₃-C₁₀ cycloalkyl or 3- to 10-membered heterocycloalkyl.

In some embodiments, R¹ is C₃-C₁₀ cycloalkyl or 3- to 10-membered heterocycloalkyl, wherein the cycloalkyl or heterocycloalkyl is optionally substituted with one or more R^{X}.

In some embodiments, R¹ is C₃-C₁₀ cycloalkyl or 3- to 10-membered heterocycloalkyl, wherein the cycloalkyl or heterocycloalkyl is optionally substituted with one, two, three, four, five, or six R^{X}.

In some embodiments, R¹ is unsubstituted C₃-C₁₀ cycloalkyl.

In some embodiments, R¹ is C₃-C₁₀ cycloalkyl, wherein the cycloalkyl is optionally substituted with one, two, three, four, five, or six R).

In some embodiments, R¹ is C₃-C₁₀ cyclohexyl optionally substituted with one R^{X}.

In some embodiments, R¹ is cyclohexyl optionally substituted with two R^{X}.

In some embodiments, R¹ is cyclohexyl optionally substituted with three R^{X}.

In some embodiments, R¹ is cyclohexyl optionally substituted with four R^{X}.

In some embodiments, R¹ is cyclohexyl optionally substituted with five R^{X}.

In some embodiments, R¹ is 3- to 10-membered heterocycloalkyl optionally substituted with one or more R^{X}.

In some embodiments, R¹ is unsubstituted 3- to 10-membered heterocycloalkyl.

In some embodiments, each RN independently is halogen, cyano, —OR^{X1}, —N(R^{X1})₂, oxo, C₁-C₁₀ alkyl, or C₃-C₁₀ cycloalkyl.

In some embodiments, each R^{X} independently is —OR^{X1}, —N(R^{X1})₂, or oxo.

In some embodiments, at least one R^{X} is halogen.

In some embodiments, at least one R^{X} is Br.

In some embodiments, at least one R^{X} is Cl.

In some embodiments, at least one RN is F.

In some embodiments, at least one R^{X} is I.

In some embodiments, at least one R^{X} is cyano.

In some embodiments, at least one R^{X} is —OR^{X1} (e.g., —OH or —O(C₁-C₁₀ alkyl)).

In some embodiments, at least one R^{X} is —N(R^{X1})₂ (e.g., —NH₂, —NH(C₁-C₁₀ alkyl), or —N(C₁-C₁₀ alkyl)₂).

In some embodiments, at least one R^{X} is oxo.

In some embodiments, at least one R^{X} is C₁-C₁₀ alkyl.

In some embodiments, at least one R^{X} is C₃-C₁₀ cycloalkyl.

In some embodiments, R¹ is substituted by one halogen.

In some embodiments, R¹ is substituted by one Br.

In some embodiments, R¹ is substituted by one Cl.

In some embodiments, R¹ is substituted by one F.

In some embodiments, R¹ is substituted by one I.

In some embodiments, R¹ is substituted by one cyano.

In some embodiments, R¹ is substituted by one —OR^{X1} (e.g., —OH or —O(C₁-C₁₀ alkyl)).

In some embodiments, R¹ is substituted by one —N(R^{X1})₂ (e.g., —NH₂, —NH(C₁-C₁₀ alkyl), or —N(C₁-C₁₀ alkyl)₂).

In some embodiments, R¹ is substituted by one oxo.

In some embodiments, R¹ is substituted by one C₁-C₁₀ alkyl.

In some embodiments, R¹ is substituted by one C₃-C₁₀ cycloalkyl.

In some embodiments, at least one R^{X1} is H.

In some embodiments, each R^{X1} is H.

In some embodiments, at least one R^{X1} is C₁-C₆ alkyl.

In some embodiments, each R^{X1} is C₁-C₆ alkyl.

In some embodiments, R¹ is trans-4-hydroxycyclohexyl.

In some embodiments, R¹ is cis-4-hydroxycyclohexyl.

In some embodiments, R² is phenyl optionally substituted with one or more R^{Y}.

In some embodiments, each R^{Y} independently is halogen, cyano, —OR^{Y1}, —N(R^{Y1})₂, C₁-C₁₀ alkyl, or C₃-C₁₀ cycloalkyl.

In some embodiments, at least one R^{Y} is halogen.

In some embodiments, at least one R^{Y} is Br.

In some embodiments, at least one R^{Y} is Cl.

In some embodiments, at least one R^{Y} is F.

In some embodiments, at least one R^{Y} is I.

In some embodiments, at least one R^{Y} is cyano.

In some embodiments, at least one R^{Y} is —OR^{Y1} (e.g., —OH or —O(C₁-C₁₀ alkyl)).

In some embodiments, at least one R^{Y} is —N(R^{Y1})₂ (e.g., —NH₂, —NH(C₁-C₁₀ alkyl), or —N(C₁-C₁₀ alkyl)₂).

In some embodiments, at least one R^{Y} is oxo.

In some embodiments, at least one R^{Y} is C₁-C₁₀ alkyl.

In some embodiments, at least one R^{Y} is C₃-C₁₀ cycloalkyl.

In some embodiments, at least one R^{Y1} is H.

In some embodiments, each R^{Y1} is H.

In some embodiments, at least one R^{Y1} is C₁-C₆ alkyl.

In some embodiments, each R^{Y1} is C₁-C₆ alkyl.

In some embodiments. X is absent or CH₂. In some embodiments, X is absent. In some embodiments, X is CH₂.

In some embodiments, Z is CH₂, (CH₂)₂, or (CH₂)₃. In some embodiments, Z is CH₂. In some embodiments, Z is (CH₂)₂. In some embodiments, Z is (CH₂)₃.

In some embodiments, Y is cyclopentyl or cyclohexyl. In some embodiments, Y is cyclopentyl. In some embodiments, Y is cyclohexyl.

In some embodiments, a is 0 or 1. In some embodiments, a is 0. In some embodiments, a is 1.

In some embodiments, b is 1, 2, or 3. In some embodiments, b is 1. In some embodiments, b is 2. In some embodiments, b is 3.

In some embodiments, Y is 1,3-cyclopentyl, 1,3-cyclohexyl, or 1,4-cyclohexyl. In some embodiments, Y is 1,3-cyclopentyl. In some embodiments, 1,3-cyclohexyl. In some embodiments, Y is or 1,4-cyclohexyl. 1,3-cyclopentyl 1,3-cyclohexyl 1,4-cyclohexyl

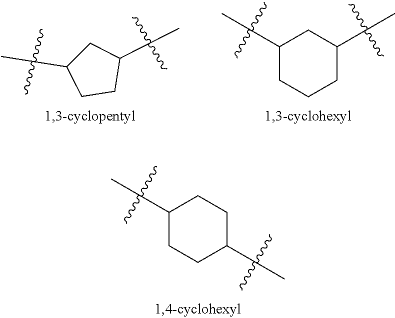 [SMILES: *C1CCC(*)C1 ; *C1CCCC(*)C1 ; *C1CCC(*)CC1]

In some embodiments, Y is 1,3-cyclopentyl, a is 0, and b is 1. In some embodiments, Y is 1,3-cyclopentyl, a is 0, and b is 2. In some embodiments, Y is 1,3-cyclopentyl, a is 0, and b is 3.

In some embodiments, Y is 1,3-cyclohexyl, a is 0, and b is 1. In some embodiments, Y is 1,3-cyclohexyl, a is 0, and b is 2. In some embodiments, Y is 1,3-cyclohexyl, a is 0, and b is 3.

In some embodiments, Y is 1,4-cyclohexyl, a is 0, and b is 1. In some embodiments, Y is 1,4-cyclohexyl, a is 0, and b is 2. In some embodiments, Y is 1,4-cyclohexyl, a is 0, and b is 3.

In some embodiments, Y is 1,3-cyclopentyl, a is 1, and b is 1. In some embodiments, Y is 1,3-cyclopentyl, a is 1, and b is 2. In some embodiments, Y is 1,3-cyclopentyl, a is 1, and b is 3.

In some embodiments, Y is 1,3-cyclohexyl, a is 1, and b is 1. In some embodiments,
Y is 1,3-cyclohexyl, a is 1, and b is 2. In some embodiments, Y is 1,3-cyclohexyl, a is 1, and b is 3.

In some embodiments, Y is 1,4-cyclohexyl, a is 1, and b is 1. In some embodiments, is 1,4-cyclohexyl, a is I, and b is 2. In some embodiments, Y is 1,4-cyclohexyl, a is 1, and b is 3.

In some embodiments, the compound has a structure represented by:

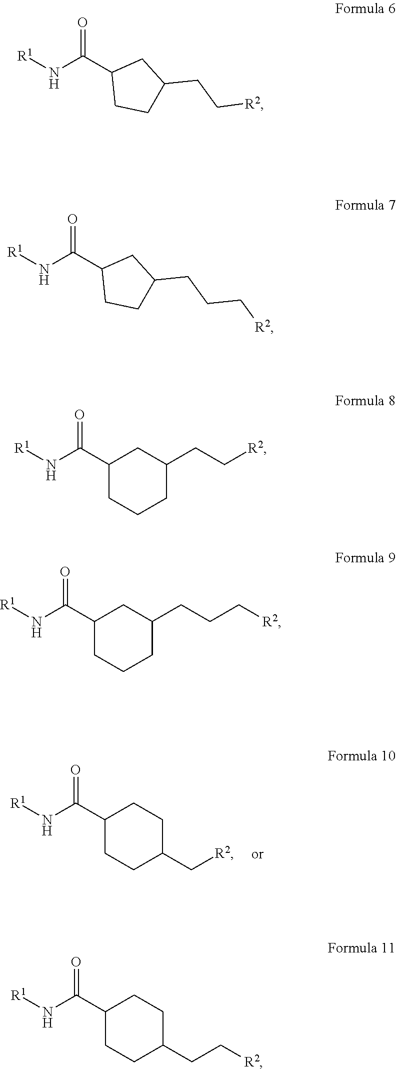

or a pharmaceutically acceptable salt thereof.

In some embodiments, the compound has a structure represented by:

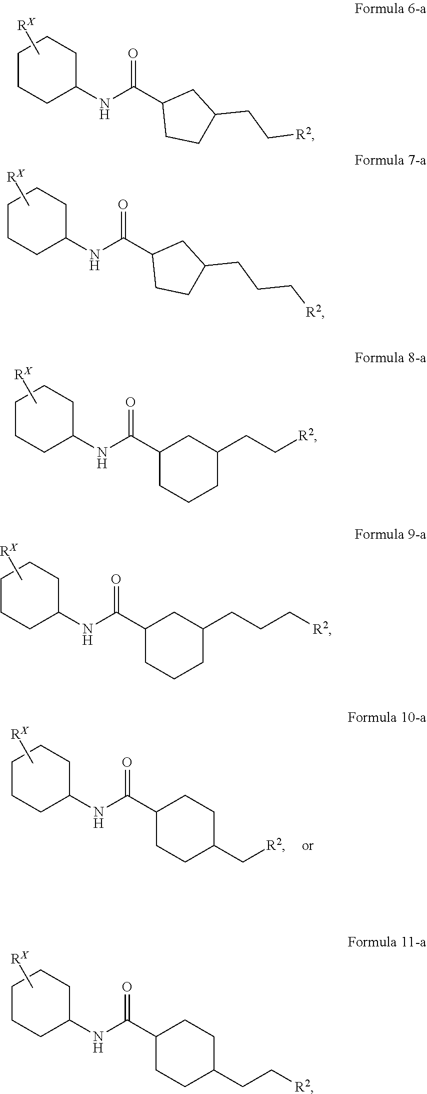

or a pharmaceutically acceptable salt thereof.

In some embodiments, the compound has a structure represented by:

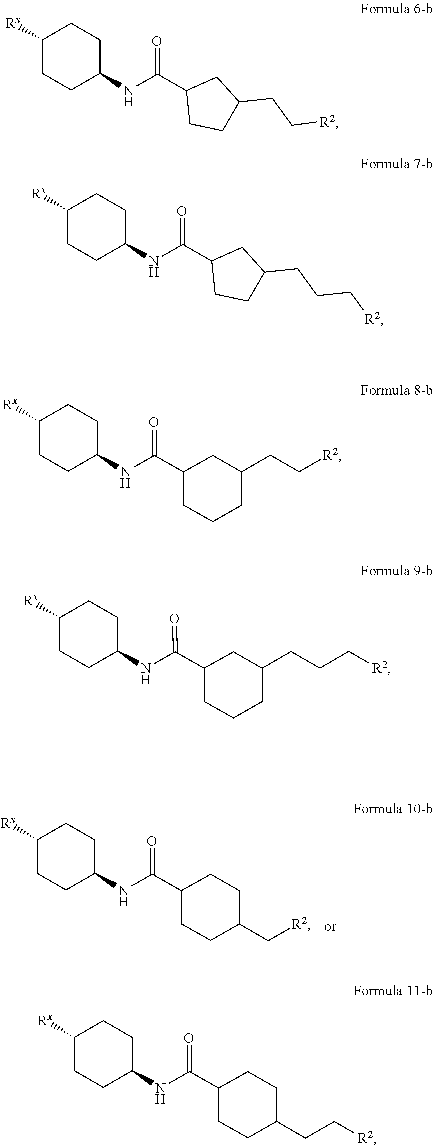

or a pharmaceutically acceptable salt thereof.

In some embodiments, the compound has a structure represented by:

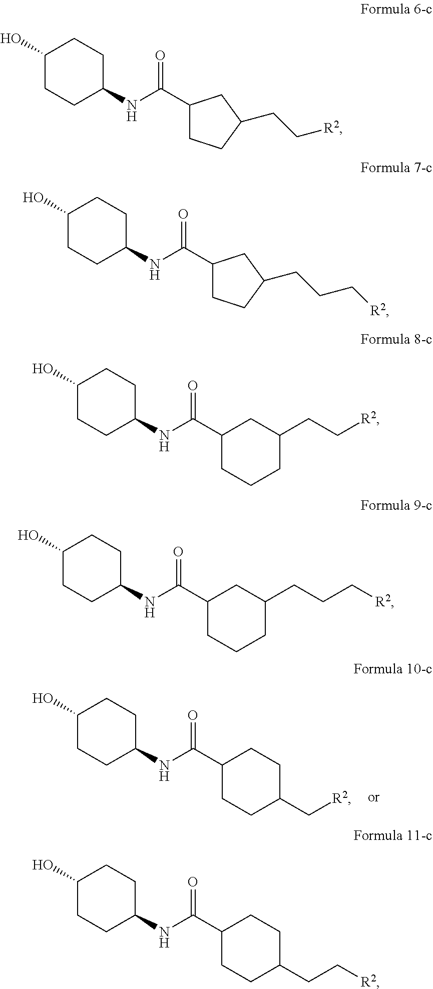

or a pharmaceutically acceptable salt thereof.

In some embodiments, the compound has a structure represented by:

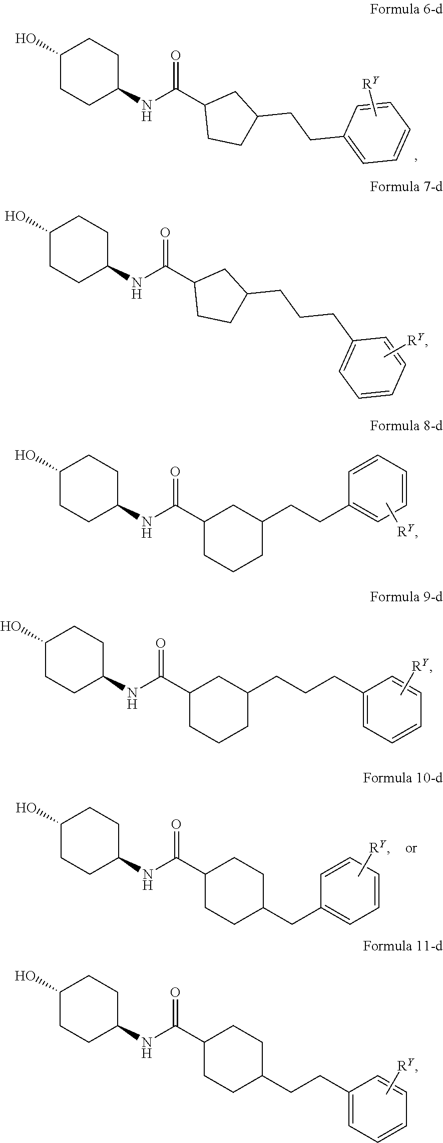

or a pharmaceutically acceptable salt thereof.

In some embodiments, Y is 1,2-cyclopentyl or 1,2-cyclohexyl. In some embodiments, Y is 1,2-cyclohexyl.

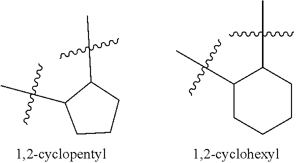 [SMILES: *C1CCCC1* ; *C1CCCCC1*]

In some embodiments, Y is 1,2-cyclopentyl, a is 0, and b is I. In some embodiments, Y is 1,2-cyclopentyl, a is 0, and b is 2. In some embodiments, Y is 1,2-cyclopentyl, a is 0, and b is 3.

In some embodiments, Y is 1,2-cyclohexyl, a is 0, and b is 1. In some embodiments, Y is 1,2-cyclohexyl, a is 0, and b is 2. In some embodiments, Y is 1,2-cyclohexyl, a is 0, and b is 3.

In some embodiments, Y is 1,2-cyclopentyl, a is 1, and b is 1. In some embodiments, Y is 1,2-cyclopentyl, a is 1, and b is 2. In some embodiments, Y is 1,2-cyclopentyl, a is 1, and b is 3.

In some embodiments, Y is 1,2-cyclohexyl, a is 1, and b is 1. In some embodiments, Y is 1,2-cyclohexyl, a is 1, and b is 2. In some embodiments, Y is 1,2-cyclohexyl, a is 1, and b is 3.

In some embodiments, the compound has a structure represented by:

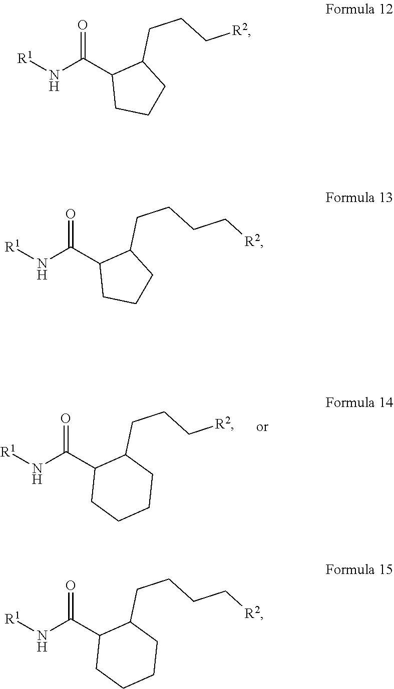

or a pharmaceutically acceptable salt thereof.

In some embodiments, the compound has a structure represented by:

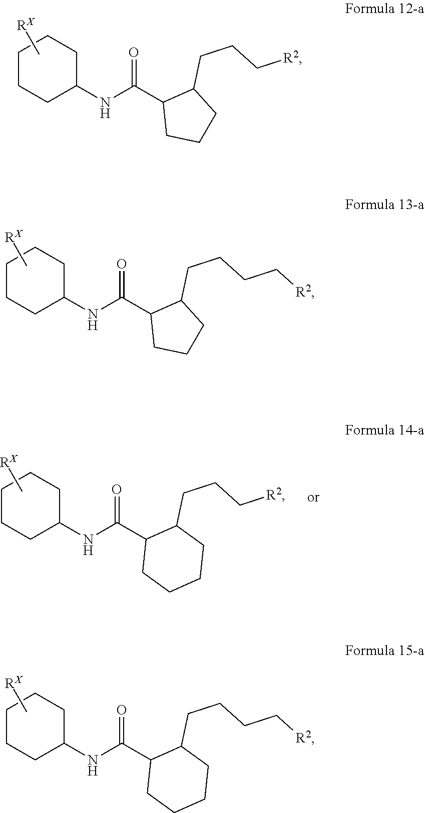

or a pharmaceutically acceptable salt thereof.

In some embodiments, the compound has a structure represented by:

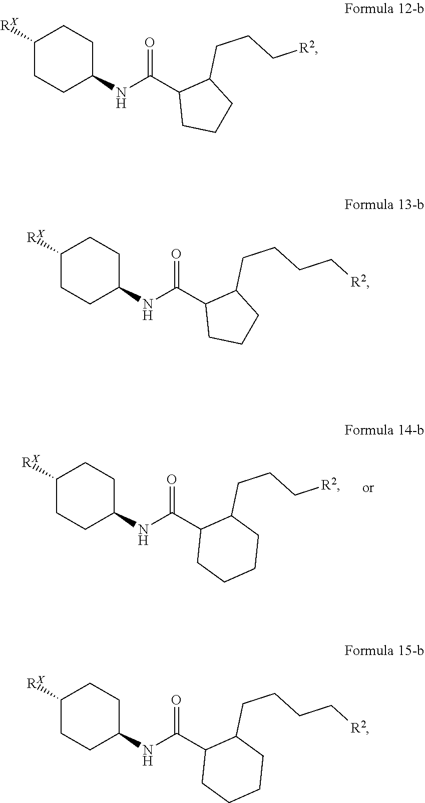

or a pharmaceutically acceptable salt thereof.

In some embodiments, the compound has a structure represented by:

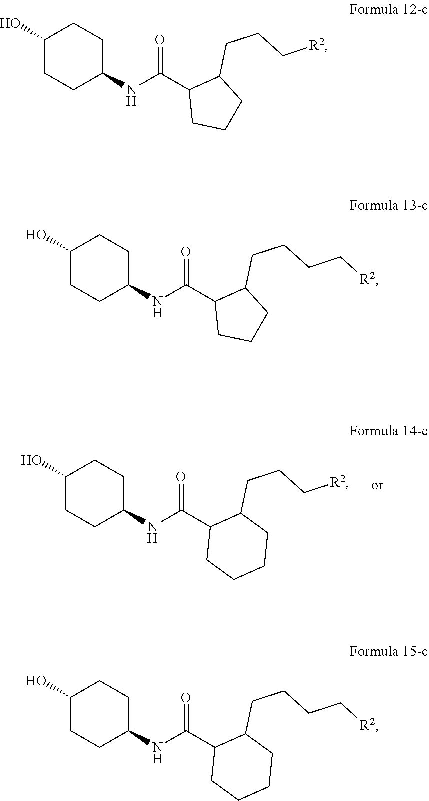

or a pharmaceutically acceptable salt thereof.

In some embodiments, the compound has a structure represented by:

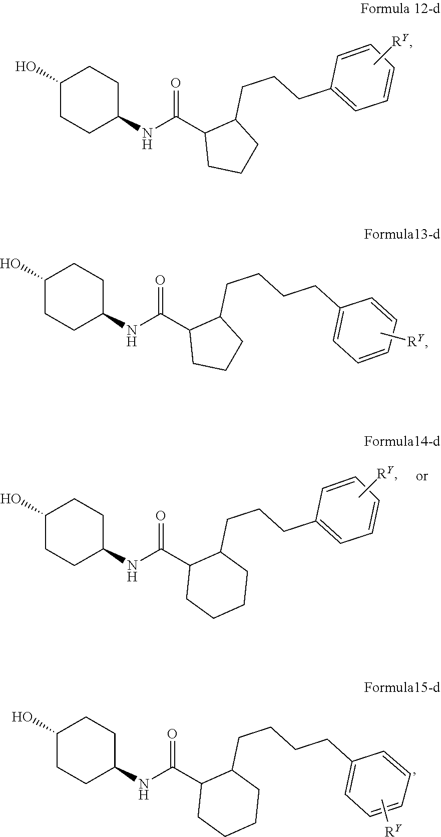

or a pharmaceutically acceptable salt thereof.

In some embodiments, the compound is selected from the group consisting of:

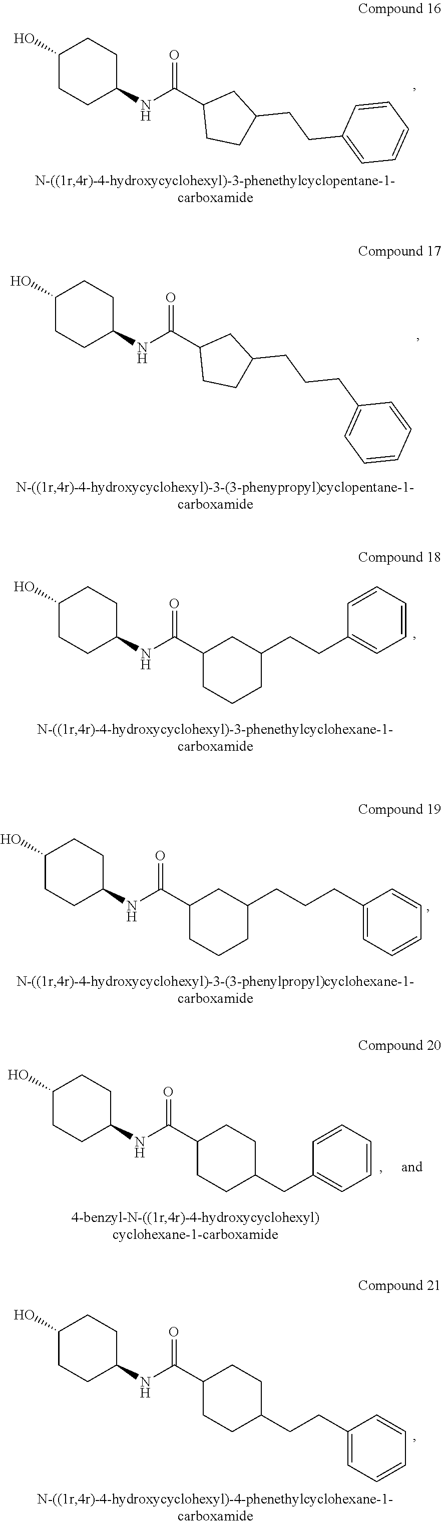 [SMILES: O=C(N[C@H]1CC[C@H](O)CC1)C1CCC(CCc2ccccc2)C1 ; O=C(N[C@H]1CC[C@H](O)CC1)C1CCC(CCCc2ccccc2)C1 ; O=C(N[C@H]1CC[C@H](O)CC1)C1CCCC(CCc2ccccc2)C1 ; O=C(N[C@H]1CC[C@H](O)CC1)C1CCCC(CCCc2ccccc2)C1 ; O=C(N[C@H]1CC[C@H](O)CC1)C1CCC(Cc2ccccc2)CC1 ; O=C(N[C@H]1CC[C@H](O)CC1)C1CCC(CCc2ccccc2)CC1]

or a pharmaceutically acceptable salt thereof.

It is understood that, advantageously, when R¹ is trans-4-hydroxycyclohexyl, the trans-stereochemistry of the 4-hydroxycyclohexyl group and the (R,R)-stereochemistry of the cyclopropane ring may be established before assembling the mitofusin activators together.

As such, the mitofusin activators may exhibit high stereoisomeric purity. In some embodiments, the compound is of greater than a 1:1 molar ratio of the (R,R) configuration relative to the (S,S) configuration of the cyclopropane ring. In some embodiments, the compound is of about 60% or greater (RR) configuration, or about 70% or greater (R,R) configuration, or about 80% or greater (R,R) configuration, or about 90% or greater (R,R) configuration, or about 95% or greater (R,R) configuration, or about 97% or greater (R,R) configuration, or about 99% or greater (R,R) configuration, or about 99.9% or greater (R,R) configuration. In some embodiments, the compound is of an enantiomerically pure (R,R) configuration of the cyclopropane ring.

In some embodiments, the compound (e.g., Compounds 16-21) is of about 10% enantiomeric excess (“ee”) or greater, about 20% ee or greater, about 30% ee or greater, about 40% ee or greater, about 50% ee or greater, about 60% ee or greater, about 70% ee or greater, about 80% ee or greater, about 90% ee or greater, about 95% ee or greater, about 96% ee or greater, about 97% ee or greater, about 98% ee or greater, about 99% ee or greater, about 99.5% ee or greater, or about 99.9% ee or greater.

In some aspects, the present disclosure provides a compound being an isotopic derivative (e.g., isotopically labeled compound) of any one of the compounds disclosed herein.

It is understood that the isotopic derivative can be prepared using any of a variety of art-recognized techniques. For example, the isotopic derivative can generally be prepared by carrying out the procedures disclosed in the Schemes and/or in the Examples described herein, by substituting an isotopically labeled reagent for a non-isotopically labeled reagent.

In some embodiments, the isotopic derivative is a deuterium labeled compound.

In some embodiments, the isotopic derivative is a deuterium labeled compound of any one of the compounds of the Formulae disclosed herein.

It is understood that the deuterium labeled compound comprises a deuterium atom having an abundance of deuterium that is substantially greater than the natural abundance of deuterium, which is 0.015%.

In some embodiments, the deuterium labeled compound has a deuterium enrichment factor for each deuterium atom of at least 3500 (52.5% deuterium incorporation at each deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). As used herein, the term “deuterium enrichment factor” means the ratio between the deuterium abundance and the natural abundance of a deuterium.

It is understood that the deuterium labeled compound can be prepared using any of a variety of art-recognized techniques. For example, the deuterium labeled compound can generally be prepared by carrying out the procedures disclosed in the Schemes and/or in the Examples described herein, by substituting a deuterium labeled reagent for a non-deuterium labeled reagent.

A compound of the present disclosure or a pharmaceutically acceptable salt or solvate thereof that contains the aforementioned deuterium atom(s) is within the scope of the disclosure. Further, substitution with deuterium (i.e., ²H) may afford certain therapeutic advantages resulting from greater metabolic stability, e.g., increased in vivo half-life or reduced dosage requirements.

For the avoidance of doubt, it is to be understood that, where in this specification a group is qualified by “described herein”, the said group encompasses the first occurring and broadest definition as well as each and all of the particular definitions for that group.

A suitable pharmaceutically acceptable salt of a compound of the disclosure is, for example, an acid-addition salt of a compound of the disclosure which is sufficiently basic, for example, an acid-addition salt with, for example, an inorganic or organic acid, for example hydrochloric, hydrobromic, sulphuric, phosphoric, trifluoroacetic, formic, citric methane sulphonate or maleic acid. In addition, a suitable pharmaceutically acceptable salt of a compound of the disclosure which is sufficiently acidic is an alkali metal salt, for example a sodium or potassium salt, an alkaline earth metal salt, for example a calcium or magnesium salt, an ammonium salt or a salt with an organic base which affords a pharmaceutically acceptable cation, for example a salt with methylamine, dimethylamine, diethylamine, trimethylamine, piperidine, morpholine or tris-(2-hydroxyethyl)amine.

It will be understood that the compounds of the present disclosure and any pharmaceutically acceptable salts thereof, comprise stereoisomers, mixtures of stereoisomers, polymorphs of all isomeric forms of said compounds.

As used herein, the term “isomerism” means compounds that have identical molecular formulae but differ in the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images of each other are termed “enantiomers” or sometimes optical isomers. A mixture containing equal amounts of individual enantiomeric forms of opposite chirality is termed a “racemic mixture.”

As used herein, the term “chiral center” refers to a carbon atom bonded to four nonidentical substituents.

As used herein, the term “chiral isomer” means a compound with at least one chiral center. Compounds with more than one chiral center may exist either as an individual diastereomer or as a mixture of diastereomers, termed “diastereomeric mixture.” When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al. Angew. Chem. Inter. Edit. 1966, 5, 385; errata 511; Cahn et al., Angew. Chen. 1966, 78, 413; Cahn and Ingold, J. Chem. Soc. 1951 (London), 612; Cahn et al., Experientia 1956, 12, 81; Cahn, J. Chem. Educ. 1964, 41, 116).

As used herein, the term “geometric isomer” means the diastereomers that owe their existence to hindered rotation about double bonds or a cycloalkyl linker (e.g., 1,3-cyclobutyl). These configurations are differentiated in their names by the prefixes cis and trans, or Z and E, which indicate that the groups are on the same or opposite side of the double bond in the molecule according to the Cahn-Ingold-Prelog rules.

It is to be understood that the compounds of the present disclosure may be depicted as different chiral isomers or geometric isomers. It is also to be understood that when compounds have chiral isomeric or geometric isomeric forms, all isomeric forms are intended to be included in the scope of the present disclosure, and the naming of the compounds does not exclude any isomeric forms, it being understood that not all isomers may have the same level of activity.

It is to be understood that the structures and other compounds discussed in this disclosure include all atropic isomers thereof. It is also to be understood that not all atropic isomers may have the same level of activity.

As used herein, the term “atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques, it has been possible to separate mixtures of two atropic isomers in select cases.

As used herein, the term “tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerisation is possible, a chemical equilibrium of the tautomers will be reached. The exact ratio of the tautomers depends on several factors, including temperature, solvent and pH. The concept of tautomers that are interconvertible by tautomerisations is called tautomerism. Of the various types of tautomerism that are possible, two are commonly observed. In keto-enol tautomerism a simultaneous shift of electrons and a hydrogen atom occurs, Ring-chain tautonerism arises as a result of the aldehyde group (-110) in a sugar chain molecule reacting with one of the hydroxy groups (—OH) in the same molecule to give it a cyclic (ring-shaped) form as exhibited by glucose.

It is to be understood that the compounds of the present disclosure may be depicted as different tautomers. It should also be understood that when compounds have tautomeric forms, all tautomeric forms are intended to be included in the scope of the present disclosure, and the naming of the compounds does not exclude any tautomer form. It will be understood that certain tautomers may have a higher level of activity than others.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarised light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this disclosure may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the disclosure may have geometric isomeric centers (E- and Z-isomers). It is to be understood that the present disclosure encompasses all optical, diastereoisomers and geometric isomers and mixtures thereof that possess inflammasome inhibitory activity.

The present disclosure also encompasses compounds of the disclosure as defined herein which comprise one or more isotopic substitutions.

It is to be understood that the compounds of any Formula described herein include the compounds themselves, as well as their salts, and their solvates, if applicable. A salt, for example, can be formed between an anion and a positively charged group (e.g., amino) on a substituted compound disclosed herein. Suitable anions include chloride, bromide, iodide, sulphate, bisulphate, sulphamate, nitrate, phosphate, citrate, methanesulphonate, trifluoroacetate, glutamate, glucuronate, glutarate, malate, maleate, succinate, fumarate, tartrate, tosylate, salicylate, lactate, naphthalenesulphonate, and acetate (e.g., trifluoroacetate).

As used herein, the term “pharmaceutically acceptable anion” refers to an anion suitable for forming a pharmaceutically acceptable salt. Likewise, a salt can also be formed between a cation and a negatively charged group (e.g., carboxylate) on a substituted compound disclosed herein. Suitable cations include sodium ion, potassium ion, magnesium ion, calcium ion, and an ammonium cation such as tetramethylammonium ion or diethylamine ion. The substituted compounds disclosed herein also include those salts containing quaternary nitrogen atoms.

It is to be understood that the compounds of the present disclosure, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

As used herein, the term “solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound.

As used herein, the term “derivative” refers to compounds that have a common core structure and are substituted with various groups as described herein.

As used herein, the term “bioisostere” refers to a compound resulting from the exchange of an atom or of a group of atoms with another, broadly similar, atom or group of atoms. The objective of a bioisosteric replacement is to create a new compound with similar biological properties to the parent compound. The bioisosteric replacement may be physicochemically or topologically based. Examples of carboxylic acid bioisosteres include, but are not limited to, acyl sulphonamides, tetrazoles, sulphonates and phosphonates. See, e.g., Patani and LaVoie, Chem. Rev. 96, 3147-3176, 1996.

It is also to be understood that certain compounds of the present disclosure may exist in solvated as well as unsolvated forms such as, for example, hydrated forms. A suitable pharmaceutically acceptable solvate is, for example, a hydrate such as hemi-hydrate, a mono-hydrate, a di-hydrate or a tri-hydrate.

### Synthesis of the Compounds

It is understood that the deuterium labeled compound can be prepared using any of a variety of art-recognized techniques. For example, the deuterium labeled compound can generally be prepared by carrying out the procedures disclosed in the Schemes and/or in the Examples described herein, by substituting a deuterium labeled reagent for a non-deuterium labeled reagent.

In some aspects, the present disclosure provides a method of preparing a compound disclosed herein.

In some aspects, the present disclosure provides a method of preparing a compound, comprising one or more steps as described herein.

In some aspects, the present disclosure provides a compound obtainable by, or obtained by, or directly obtained by a method for preparing a compound described herein.

In some aspects, the present disclosure provides an intermediate being suitable for use in a method for preparing a compound described herein.

In some embodiments, a compound of described herein is prepared according to Scheme A below.

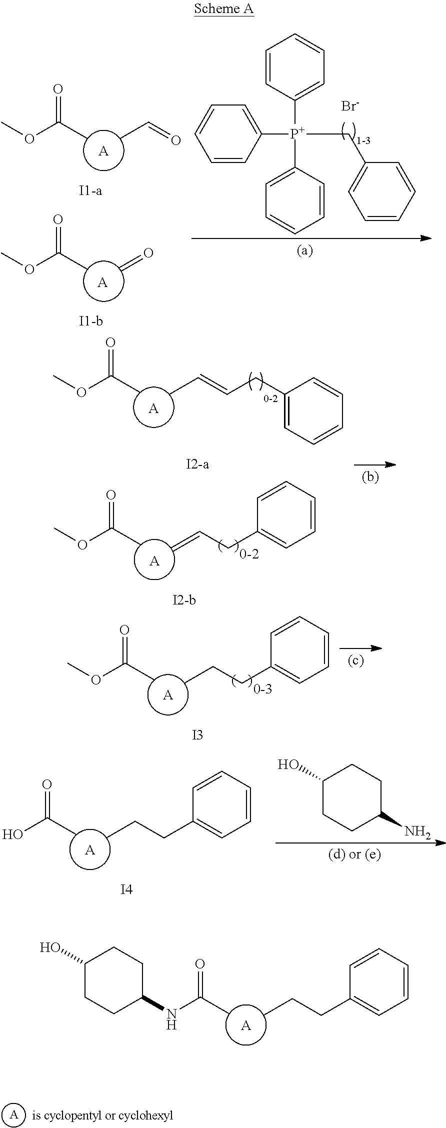

In some embodiments, the synthesis in Scheme A is performed with one or more of the following conditions:
(a) t-BuOK, THF, −10° C., 2 hrs
(b) H₂, Pd/C, MeOH, RT, 2 hrs
(d) T₃P, DIEA, DCM, RT, 12 bra
(e) HATU, TEA, THF

The compounds of the present disclosure can be prepared by any suitable technique known in the art. Particular processes for the preparation of these compounds are described further in the accompanying examples.

In the description of the synthetic methods described herein and in any referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilized.

It will be appreciated that during the synthesis of the compounds of the disclosure in the processes defined herein, or during the synthesis of certain starting materials, it may be desirable to protect certain substituent groups to prevent their undesired reaction. The skilled chemist will appreciate when such protection is required, and how such protecting groups may be put in place, and later removed. For examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule. Thus, if reactants include, for example, groups such as amino, carboxy or hydroxy it may be desirable to protect the group in some of the reactions mentioned herein.

The resultant compounds of the present disclosure can be isolated and purified using techniques well known in the art.

Moreover, by utilizing the procedures described herein, in conjunction with ordinary skills in the art, additional compounds of the present disclosure can be readily prepared. Those skilled in the art will readily understand that known variations of the conditions and processes of the following preparative procedures can be used to prepare these compounds.

As will be understood by the person skilled in the art of organic synthesis, compounds of the present disclosure are readily accessible by various synthetic routes, some of which are exemplified in the accompanying examples. The skilled person will easily recognize which kind of reagents and reactions conditions are to be used and how they are to be applied and adapted in any particular instance—wherever necessary or useful—in order to obtain the compounds of the present disclosure. Furthermore, some of the compounds of the present disclosure can readily be synthesized by reacting other compounds of the present disclosure under suitable conditions, for instance, by converting one particular functional group being present in a compound of the present disclosure, or a suitable precursor molecule thereof, into another one by applying standard synthetic methods, like reduction, oxidation, addition or substitution reactions; those methods are well known to the skilled person. Likewise, the skilled person will apply—whenever necessary or useful—synthetic protecting (or protective) groups; suitable protecting groups as well as methods for introducing and removing them are well-known to the person skilled in the art of chemical synthesis and are described, in more detail, in, e.g., P. G. M. Wuts, T. W. Greene, “Greene's Protective Groups in Organic Synthesis”, 4th edition (2006) (John Wiley & Sons).

### Pharmaceutical Compositions

In another exemplary aspect, the disclosure provides pharmaceutical compositions comprising any compound herein, or a pharmaceutically acceptable form thereof. In some embodiments, a pharmaceutical composition comprises a therapeutically effective amount of any compound described herein, or any pharmaceutically acceptable form thereof.

in some embodiments, a pharmaceutically acceptable form of a compound includes any pharmaceutically acceptable salts, hydrates, solvates, isomers, prodrugs, and isotopically labeled derivatives thereof.

In some embodiments, a pharmaceutical composition comprises any compound described herein, or a pharmaceutically acceptable salt thereof.

In some embodiments, a pharmaceutical composition comprises a pharmaceutically acceptable excipient.

For the purposes of the present invention the term “excipient” and “carrier” are used interchangeably throughout the description of the present invention and said terms are defined herein as, “ingredients which are used in the practice of formulating a safe and effective pharmaceutical composition.”

The formulator will understand that excipients are used primarily to serve in delivering a safe, stable, and functional pharmaceutical, serving not only as part of the overall vehicle for delivery but also as a means for achieving effective absorption by the recipient of the active ingredient. An excipient may fill a role as simple and direct as being an inert filler, or an excipient as used herein may be part of a pH stabilizing system or coating to insure delivery of the ingredients safely to the stomach. The formulator can also take advantage of the fact the compounds of the present invention have improved cellular potency, pharmacokinetic properties, as well as improved oral bioavailability.

Accordingly, in some embodiments, provided herein are pharmaceutical compositions comprising one or more compounds as disclosed herein, or a pharmaceutically acceptable form thereof (e.g., pharmaceutically acceptable salts, hydrates, solvates, isomers, prodrugs, and isotopically labeled derivatives), and one or more pharmaceutically acceptable excipients, carriers, including inert solid diluents and fillers, diluents, including sterile aqueous solution and various organic solvents, permeation enhancers, solubilizers and adjuvants. In some embodiments, a pharmaceutical composition described herein includes a second active agent such as an additional therapeutic agent, (e.g., a chemotherapeutic).

Accordingly, the present teachings also provide pharmaceutical compositions that include at least one compound described herein, or any pharmaceutically salt thereof, and one or more pharmaceutically acceptable carriers, excipients, or diluents. Examples of such carriers are well known to those skilled in the art and can be prepared in accordance with acceptable pharmaceutical procedures, such as, for example, those described in Remington's Pharmaceutical Sciences, 17th edition, ed. Alfonoso R., Gennaro, Mack Publishing Company, Easton, PA (1985), the entire disclosure of which is incorporated by reference herein for all purposes. As used herein, “pharmaceutically acceptable” refers to a substance that is acceptable tor use in pharmaceutical applications from a toxicological perspective and does not adversely interact with the active ingredient. Accordingly, pharmaceutically acceptable carriers are those that are compatible with the other ingredients in the composition and are biologically acceptable. Supplementary active ingredients can also be incorporated into the pharmaceutical compositions.

Compounds of the present teachings can be administered orally or parenterally, neat or in combination with conventional pharmaceutical carriers. Applicable solid carriers can include one or more substances which can also act as flavoring agents, lubricants, solubilizers, suspending agent, fillers, glidants, compression aids, binders or tablet-disintegrating agents, or encapsulating materials. Pharmaceutical compositions in the form of oral formulations containing a compound disclosed herein can comprise any Conventionally used oral form, including tablets, capsules, buccal forms, troches, lozenges and oral liquids, suspensions or solutions. In powders, the carrier can be a finely divided solid, which is an admixture with a finely divided compound. In tablets, a compound disclosed herein can be mixed with a carrier having die necessary compression properties in suitable proportions and compacted in the shape and size desired. The powders and tablets can contain up to 99% of the compound.

Capsules can contain mixtures of one or mire compound(s) disclosed herein with inert filler(s) and/or diluent(s) such as pharmaceutically acceptable starches (e.g. corn, potato or tapioca starch), sugars, artificial sweetening agents, powdered celluloses (e.g. crystalline and microcrystalline celluloses), flours, gelatins, gums, and the like.

Useful tablet formulations can be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, sodium lauryl sulfate, tale, sugars, lactose, dextrin, starch, gelatin, cellulose, methyl cellulose, microcrystalline cellulose, sodium carboxymethyl, cellulose, carboxymethylcellulose calcium, polyvinylpyrrolidine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes, and ion exchange resins. Surface modifying agents include nonionic and anionic surface modifying agents. Representative examples of surface modifying agent include but are not limited to, poloxamer 188, benzalkonium chloride, calcium stearate, cetostearl alcohol, cetomacrogol emulsifying wax, sorbitan esters, colloidal silicon dioxide, phosphates, sodium dodeclsulfate, magnesium aluminum silicate, and triethanolamine. Oral formulations described herein can utilize standard delay or time-release formulations to alter the absorption of the compound(s). An oral formulation can also consist of administering a compound disclosed herein in water or fruit juice, containing appropriate solubilizers or emulsifiers as needed.

Liquid carriers can be used in preparing solutions, suspensions, emulsions, syrups, elixirs, and for inhaled delivery. A compound of the present teachings can be dissolved or suspended in a pharmaceutically acceptable liquid carrier such as water, an organic solvent, or a mixture of both, or a pharmaceutically acceptable oils or fats. The liquid carrier can contain other suitable pharmaceutical additives such as solubilizers, emulsifiers, buffers, preservatives, sweeteners flavoring agents, suspending agents, thickening agents, colors, viscosity regulators, stabilizers, and osmo-regulators. Examples of liquid carriers for oral and parenteral administration include, but are not limited to, water (particularly containing additives as described herein, e.g., cellulose derivatives such as a sodium carboxymethyl cellulose solution), alcohols (including monohydric alcohols and polyhydric alcohols, e.g., glycols) and their derivatives, and oils (e.g., fractionated coconut oil and arachis oil). For parenteral administration, the carrier can be an oily ester such as ethyl oleate and isopropyl myristate Sterile liquid carriers are used in sterile liquid form compositions for parenteral administration. The liquid carrier for pressurized compositions can be halogenated hydrocarbon or other pharmaceutically acceptable propellants.

Liquid pharmaceutical compositions, which are sterile solutions or suspensions, can be utilized by, for example, intramuscular, intraperitoneal or subcutaneous injection. Sterile solutions can also be administered intravenously. Compositions for oral administration can be in either liquid or solid form.

In some embodiments, a pharmaceutical composition is in unit dosage form, for example, as tablets, capsules, powders, solutions, suspensions, emulsions, granules, or suppositories. In such form, the pharmaceutical composition can be sub-divided in unit dose(s) containing appropriate quantities of the compound. The unit dosage forms can be packaged compositions, for example, packeted powders, vials, ampoules, prefilled syringes or sachets containing liquids. Alternatively, the unit dosage form can be a capsule or tablet itself, or it can be the appropriate number of any such compostions in package form. Such unit dosage form can contain from about 1 mg/kg of compound to about 500 mg/kg compound, and can be given in a single dose or in two or more doses. Such doses can be administered in any manner useful in directing the compound(s) to the recipient's bloodstream, including orally, via implants, parenterally (including intravenous, intraperitoneal and subcutaneous injections), rectally, virginally and transdermally.

When administered for the treatment or inhibition of a particular disease state or disorder, it is understood that an effective dosage can vary depending upon the particular compound utilized, the mode of administration, and severity of the condition being treated, as well as the various physical factors related to the individual being treated. In therapeutic applications, a compound of the present teachings can be provided to a patient already suffering from a disease in an amount sufficient to cure or at least partially ameliorate the symptoms of the disease and its complications. The dosage to be used in the treatment of a specific individual typically must be subjectively determined by the attending physician. The variables involved include the specific condition and its state as well as the size, age and response pattern of the patient.

in some cases it may be desirable to administer a compound directly to the airways of the patient, using devices such as, but not limited to, metered dose inhalers, breath-operated inhalers, multidose dry-powder inhalers, pumps, squeeze-actuated nebulized spray dispensers, aerosol dispensers, and aerosol nebulizers. For administration by intranasal or intrabronchial inhalation, the compounds of the present teachings can be formulated into a liquid composition, a solid composition, or an aerosol composition. The liquid composition can include, by way of illustration, one or more compounds of the present teachings dissolved, partially dissolved, or suspended in one or more pharmaceutically acceptable solvents and can be administered by, for example, a pump or a squeeze-actuated nebulized spray dispenser. The solvents can be, for example, isotonic saline or bacteriostatic water. The solid composition can be, by way of illustration, a powder preparation including one or more compounds of the present teachings intermixed with lactose or other inert powders that are acceptable for intrabronchial use, and can be administered by, for example, an aerosol dispenser or a device that breaks or punctures a capsule encasing the solid composition and delivers the solid composition for inhalation. The aerosol composition can include, by way of illustration, one or more compounds of the present teachings, propellants, surfactants, and co-solvents, and can be administered by, for example, a metered device. The propellants can be a chlorofluorocarbon (CFC), a hydrofluroalkane (HFA), or other propellants that are physiologically and environmentally acceptable.

Compounds described herein can be administered parenterally or intraperitoneally. Solutions or suspensions of these compounds or a pharmaceutically acceptable salts, hydrates, or esters thereof can be prepared in water suitably mixed with a surfactant such as hydroxyl-propylcellulose. Dispersions can also be prepared in glycerol, liquid polyethlene glycols, and mixtures thereof in oils. Under ordinary conditions of storage and use, these preparations typically contain a preservative to inhibit the growth of microorganisms.

The pharmaceutical forms suitable for injection, can include sterile aqueous solutions or dispersions and sterile powders for the extemporaneous preparation of sterile injectable solutions or dispersions. In some embodiments, the form can sterile and its viscosity permits it to flow through a syringe. The form preferably is stable under the conditions of manufacture and storage and can be preserved against the contaminating action of microorganisms such as bacteria and fungi. The carrier can be a solvent or dispersion medium containing, for example, water, ethanol, polyol (e.g., glycerol, propylene glycol and liquid polyethylene glycol) suitable mixtures thereof, and vegetable oils.

Compounds described herein can be administered transdermally, i.e., administered across the surface of the body and the inner linings of bodily passages including epithelial and mucosal tissues. Such administration can be carried out using the compounds of the present teachings including pharmaceutically acceptable salts, hydrates, or esters thereof, in lotions, creams, foams, patches, suspensions, solutions, and suppositories (rectal and vaginal).

Transdermal administration can be accomplished through the use of a transdermal patch containing a compound, such as a compound disclosed herein, and a carrier that can be inert to the compound, can be non-toxic to the skin, and can allow delivery of the compound for systemic absorption into the blood stream via the skin. The carrier can take any number of forms such as creams and ointments, pastes, gels, and occlusive devices. The creams and ointments can be viscous liquid or semisolid emulsions of either the oil-in-water or water-in-oil type. Pastes comprised of absorptive powders dispersed in petroleum or hydrophilic petroleum containing the compound can also be suitable. A variety of occlusive devices can be used to release the compound into the blood stream, such as a semi-permeable membrane covering a reservoir containing the compound with or without a carrier, or a matrix containing the compound Other occlusive devices are known in the literature.

Compounds described herein can be administered rectally or vaginally in the form of a conventional suppository. Suppository formulations can be made from traditional materials, including cocoa butter, with or without the addition of waxes to alter the suppository's melting point, and glycerin. Water-soluble suppository bases, such as polyethylene glycols of various molecular weights, can also be used.

Lipid formulations or nanocapsules can be used to introduce compounds of the present teachings into host cells either in vitro or in vivo. Lipid formulations and nanocapsules can be prepared by methods known in the art.

To increase the effectiveness of compounds of the present teachings, it can be desirable to combine a compound with other agents effective in the treatment of the target disease. For example, other active compounds (i.e., other active ingredients or agents) effective in treating the target disease can be administered with compounds of the present teachings. The other agents can be administered at the same time or at different times than the compounds disclosed herein.

### Kits

In some embodiments, provided herein are kits. The kits can include a compound or pharmaceutically acceptable form thereof, or pharmaceutical composition as described herein, in suitable packaging, and written material that can include instructions for use, discussion of clinical studies, listing of side effects, and the like. Kits are well suited for the delivery of solid oral dosage forms such as tablets or capsules. Such kits can also include information, such as scientific literature references, package insert materials, clinical trial results, and/or summaries of these and the like, which indicate or establish the activities and/or advantages of the pharmaceutical composition, and/or which describe dosing, administration, side effects, drug interactions, or other information useful to the health care provider. Such information can be based on the results of various studies, for example, studies using experimental animals involving in vivo models and studies based on human clinical trials,

### Methods of Use

Compounds or pharmaceutical composition of the present teachings can be useful for the treatment or prevention of a disease, disorder, or condition in a subject, for example, a human subject. The present teachings accordingly provide methods of treating or preventing a disease, disorder, or condition in a subject by providing, to a subject a compound of the present teachings (including its pharmaceutically acceptable salt) or a pharmaceutical composition that includes one or more compounds of the present teachings in combination or association with pharmaceutically acceptable carriers. Compounds of the present teachings can be administered alone or in combination with other therapeutically effective compounds or therapies for the treatment or prevention of a disease, disorder, or condition.

In some aspects, the present disclosure provides a method of treating diseases, disorders, or conditions, comprising administering to a subject in need thereof any compound described herein in a pharmaceutical composition.

In some aspects, the present disclosure provides any compound described herein in a pharmaceutical composition for use for treating diseases, disorders, or conditions, comprising administering to a subject in need thereof.

In some aspects, the present disclosure provides use of any compound described herein in a pharmaceutical composition in the manufacture of a medicament for treating diseases, disorders, or conditions, comprising administering to a subject in need thereof.

In some aspects, the present disclosure provides a method of activating mitofusin in a subject, comprising administering the compound or the pharmaceutical composition of any one of the preceding claims.

In some aspects, the present disclosure provides any compound described herein in a pharmaceutical composition for use in activating mitofusin in a subject.

In some aspects, the present disclosure provides use of the any compound described herein in a pharmaceutical composition in the manufacture of a medicament for activating mitofusin in a subject.

In some embodiments, a compound described herein, or any pharmaceutically acceptable form thereof such as a pharmaceutically acceptable salt thereof, can be used to treat or prevent a disease, disorder, or condition in a subject.

In some embodiments, a therapeutically effective amount of the compound or the pharmaceutical composition described herein is administered to the subject.

In some embodiments, the disease, disorder, or condition is associated with mitochondria.

In some embodiments, the disease, disorder, or condition is responsive to mitochondria modulation.

In some embodiments, the disease, disorder, or condition is peripheral nervous system (PNS), central nervous system (CNS) genetic or non-genetic disorder, physical damage, or chemical injury.

In some embodiments, the PNS or CNS disorder is one or more conditions selected from the group consisting of a chronic neurodegenerative condition in which mitochondrial fusion, fitness, and/or trafficking is/are impaired; a disease or disorder associated with mitofusin 1 (MFN1) or mitofusin 2 (MFN2) dysfunction; a disease associated with mitochondrial fragmentation, dysfunction, and/or dysmotility; a degenerative neuromuscular condition; Charcot-Marie-Tooth disease; Amyotrophic Lateral Sclerosis; Huntington's disease; Alzheimer's disease; Parkinson's disease; hereditary motor and sensory neuropathy; autism; autosomal dominant optic atrophy (ADOA); muscular dystrophy; Lou Gehrig's disease; cancer; mitochondrial myopathy; diabetes mellitus and deafness (DAD); Leber's hereditary optic neuropathy (LHON); Leigh syndrome; subacute sclerosing encephalopathy; neuropathy, ataxia, retinitis pigmentosa, and ptosis (NARP); myoneurogenic gastrointestinal encephalopathy (MNGIE); myoclonic epilepsy with ragged red fibers (MERRF); mitochondrial myopathy, encephalomyopathy, lactic acidosis, and stroke-like symptoms (MELAS); mtDNA depletion; mitochondrial neurogastrointestinal encephalomyopathy (MNGIE); dysautonomic mitochondrial myopathy; mitochondrial channelopathy; pyruvate dehydrogenase complex deficiency (PDCD/PDH); diabetic neuropathy; chemotherapy-induced peripheral neuropathy; crush injury; spinal cord injury (SCI); traumatic brain injury; stroke; optic nerve injury; conditions that involve axonal disconnection; and any combination thereof.

In some embodiments, the subject is human.

In some embodiments, a compound described herein, or any pharmaceutically acceptable form thereof such as a pharmaceutically acceptable salt thereof, can be used to active mitofusin in a subject (e.g., human).

### Definitions

Unless stated otherwise, the following terms and phrases as used herein are intended to have the following meanings.

The terms “treat” or “treatment”, unless otherwise indicated by context, refer to any administration of a therapeutic molecule (e.g., any compound described herein) that partially or completely alleviates, ameliorates, relieves, inhibits, reduces severity of and/or reduces incidence of one or more symptoms or features of a particular disease, disorder, and/or condition (e.g., cancer).

As used herein, the term “preventing,” “prevent,” or “protecting against” describes delaying onset or slowing progression of a disease, condition or disorder.

As used herein, the term “subject” includes human and non-human animals, as well as cell lines, cell cultures, tissues, and organs. In some embodiments, the subject is a mammal. The mammal can be e.g., a human or appropriate non-human mammal, such as primate, mouse, rat, dog, cat, cow, horse, goat, camel, sheep or a pig. The subject can also be a bird or fowl. In some embodiments, the subject is a human.

As used herein, the term “subject in need thereof” refers to a subject having a disease or having an increased risk of developing the disease. A subject in need thereof can be one who has been previously diagnosed or identified as having a disease or disorder disclosed herein. A subject in need thereof can also be one who is suffering from a disease or disorder disclosed herein. Alternatively, a subject in need thereof can be one who has an increased risk of developing such disease or disorder relative to the population at large (i.e., a subject who is predisposed to developing such disorder relative to the population at large). A subject in need thereof can have a refractory or resistant a disease or disorder disclosed herein (i.e., a disease or disorder disclosed herein that does not respond or has not yet responded to treatment), The subject may be resistant at start of treatment or may become resistant during treatment. In some embodiments, the subject in need thereof received and failed all known effective therapies for a disease or disorder disclosed herein. In some embodiments, the subject in need thereof received at least one prior therapy.

The term “therapeutically effective amount” or “effective amount” refers to an amount of a conjugate effective to treat or prevent a disease or disorder in a subject (e.g., as described herein).

As used herein, the term “pharmaceutical composition” refers to a composition in which an active agent is formulated together with one or more pharmaceutically acceptable carriers. In some embodiments, the active agent is present in unit dose amount appropriate for administration in a therapeutic regimen that shows a statistically significant probability of achieving a predetermined therapeutic effect when administered to a relevant population. In some embodiments, a pharmaceutical composition may be specially formulated for administration in solid or liquid form, including those adapted for the following oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

As used herein, the term “administration” typically refers to the administration of a composition to a subject or system to achieve delivery of an agent that is, or is included in, the composition. Those of ordinary skill in the art will be aware of a variety of routes that may, in appropriate circumstances, be utilized for administration to a subject, for example a human. Examples of routes of administration include parenteral, e.g., intravenous, intradermal, subcutaneous, oral (e.g., inhalation), transdermal (i.e., topical), transmucosal, and rectal administration. For example, in some embodiments, administration may be ocular, oral, parenteral, topical, etc. In some embodiments, administration is parenteral (e.g., intravenous administration). In some embodiments, intravenous administration is intravenous infusion. In some particular embodiments, administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e. g. intrahepatic), mucosal, nasal, oral, rectal, subcutaneous, sublingual, topical, tracheal (e.g., by intratracheal instillation), vaginal, vitreal, etc.

Unless otherwise indicated, the term “alkyl” by itself or as part of another term refers to a straight or branched, saturated hydrocarbon having the indicated number of carbon atoms (e.g., “C₁-C₈ alkyl” or “C₁-C₁₀” alkyl refer to an alkyl group having from 1 to 8 or 1 to 10 carbon atoms, respectively). When the number of carbon atoms is not indicated, the alkyl group has from 1 to 8 carbon atoms. Representative straight chain “—₁-C₈ alkyl” groups include, but are not limited to, -methyl, -ethyl, -n-propyl, -n-butyl, -n-pentyl, -n-hexyl, -n-heptyl and -n-octyl; while branched C₃-C₈ alkyls include, but are not limited to, -isopropyl, -sec-butyl, -isobutyl, -tert-butyl, -isopentyl, and -2-methylbutyl; unsaturated C₂-C₈ alkyls include, but are not limited to, -vinyl, -allyl, -1-butenyl, -2-butenyl, -isobu-tylenyl, -1 pentenyl, -2 pentenyl, -3-methyl-1-butenyl, -2 methyl-2-butenyl, -2,3 dimethyl-2-butenyl, -1-hexyl, 2-hexyl, -3-hexyl, -acetylenyl, -propynyl, -1 butynyl, -2 butynyl, —1 pentynyl, -2 pentynyl and -3 methyl 1 butynyl. Sometimes an alkyl group is unsubstituted. An alkyl group can be substituted with one or more groups. In other aspects, an alkyl group will be saturated.

As used herein, the term “optionally substituted alkyl” refers to unsubstituted alkyl or alkyl having designated substituents replacing one or more hydrogen atoms on one or more carbons of the hydrocarbon backbone. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulphydryl, alkylthio, arylthio, thiocarboxylate, sulphates, alkylsulphinyl, sulphonate, sulphamoyl, sulphonamide, nitro, trifluoromethyl, cyano, azido, heterocyclyl, alkylaryl, or an aromatic or heteroaromatic moiety.

Unless otherwise indicated, “alkylene”, by itself of as part of another term, refers to a saturated, branched or straight chain or cyclic hydrocarbon radical of the stated number of carbon atoms, typically 1-10 carbon atoms, and having two monovalent radical centers derived by the removal of two hydrogen atoms from the same or two different carbon atoms of a parent alkane. Typical alkylene radicals include, but are not limited to: methylene (—CH₂—), 1,2-ethylene (—CH₂CH₂—), 1,3-propylene (—CH₂CH₂CH₂—), 1,4-butylene (—CH₂CH₂CH₂CH₂—), and the like. In preferred aspects, an alkylene is a branched or straight chain hydrocarbon (i.e., it is not a cyclic hydrocarbon). In some embodiments, the alkylene is unsubstituted. In some embodiments, the alkylene is substituted with one or more groups.

Unless otherwise indicated, “aryl”, by itself or as part of another term, means a monovalent carbocyclic aromatic hydrocarbon radical of the stated number of carbon atoms, typically 6-20 carbon atoms, derived by the removal of one hydrogen atom from a single carbon atom of a parent aromatic ring system. Some aryl groups are represented in the exemplary structures as “Ar”. Typical aryl groups include, but are not limited to, radicals derived from benzene, substituted benzene, naphthalene, anthracene, biphenyl, and the like. An exemplary aryl group is a phenyl group. Sometimes an aryl group is unsubstituted. An aryl group can be substituted with one or more groups.

As used herein, the term “cycloalkyl” refers to a saturated hydrocarbon ring, and is exemplified by a cyclopropyl group, a cyclobutyl group, a cyclopentyl group, a cyclohexyl group, a cycloheptyl group, a cyclooctyl group, and the like. In addition to monocycloalkyl groups, polycycloalkyl groups such as bicycloalkyl groups and tricycloalkyl groups are also included. Examples of bicycloalkyl groups include norbornyl groups such as exo-2-norbornyl groups, endo-2-Tricycloalkyl groups such as norbornyl, 3-pinanyl, bicyclo [3.1.0] hexyl, bicyclo [2.2.1] heptyl, bicyclo [2.2.2] oct-2-yl Examples thereof include an adamantyl group such as a 1-adamantyl group and a 2-adamantyl group

As used herein, the term “heterocycloalkyl” refers to a saturated or partially unsaturated 3-8 membered monocyclic or 6-10 membered bicyclic (fused, bridged, or spiro) ring system having one or more heteroatoms independently selected from the group consisting of nitrogen, oxygen and sulphur, unless specified otherwise. Examples of heterocycloalkyl groups include, but are not limited to, piperidinyl, piperazinyl, pyrrolidinyl, dioxanyl, tetrahydrofuranyl, isoindolinyl, indolinyl, imidazolidinyl, pyrazolidinyl, oxazolidinyl, isoxazolidinyl, triazolidinyl, oxiranyl, azetidinyl, oxetanyl, thietanyl, 1,2,3,6-tetrahydropyridinyl, tetrahydropyranyl, dihydropyranyl, pyranyl, morpholinyl, tetrahydrothiopyranyl, 1,4-diazepanyl, 1,4-oxazepanyl, 2-oxa-5-azabicyclo[2.2.1]heptanyl, 2,5-diazabicyclo[2.2.1]heptanyl, 2-oxa-6-azaspiro[3.3]heptanyl, 2,6-diazaspiro[3.3]heptanyl, 1,4-dioxa-8-azaspiro[4.5]decanyl, 1,4-dioxaspiro[4.5]decanyl, I-oxaspiro[4.5]decanyl, I-azaspiro[4.5]decanyl, 3′H-spiro[cyclohexane-1,1′-isobenzofuran]-yl, 7′1H-spiro[cyclohexane-1,5′-furo[3,4-b]pyridin]-yl, 3′H-spiro[cyclohexane-1,1′-furo[3,4-c]pyridin]-yl, 3-azabicyclo[3.1.0]hexanyl, 3-azabicyclo[3.1.0]hexan-3-yl, 1,4,5,6-tetrabydropyrrolo[3,4-c]pyrazolyl, 3,4,5,6,7,8-hexahydropyrido[4,3-d]pyrimidinyl, 4,5,6,7-tetrahydro-1H-pyrazolo[3,4-c]pyridinyl, 5,6,7,8-tetrahydropyrido[4,3-d]pyrimidinyl, 2-azaspiro[3.3]heptanyl, 2-methyl-2-azaspiro[3.3]heptanyl, 2-azaspiro[3.5]nonanyl, 2-methyl-2-azaspiro[3.5]nonanyl, 2-azaspiro[4.5]decanyl, 2-methyl-2-azaspiro[4.5]decanyl, 2-oxa-azaspiro[3.4]octanyl, 2-oxa-azaspiro[3.4]octan-6-yl, and the like. In the case of multicyclic heterocycloalkyl, only one of the rings in the heterocycloalkyl needs to be non-aromatic.

As used herein, the term “heteroaryl” is intended to include a stable 5-, 6-, or 7-membered monocyclic or 7-, 8-, 9-, or 10-membered bicyclic aromatic heterocyclic ring which consists of carbon atoms and one or more heteroatoms independently selected from the group consisting of nitrogen, oxygen and sulphur. The nitrogen atom may be substituted or unsubstituted (i.e., N or NR wherein R is H or other substituents, as defined). The nitrogen and sulphur heteroatoms may optionally be oxidised (i.e., N→O and S(O)ₚ, where p=1 or 2). It is to be noted that total number of S and O atoms in the aromatic heterocycle is not more than 1. Examples of heteroaryl groups include pyrrole, furan, thiophene, thiazole, isothiazole, imidazole, triazole, tetrazole, pyrazole, oxazole, isoxazole, pyridine, pyrazine, pyridazine, pyrimidine, and the like.

Unless otherwise indicated, the term “heteroalkyl” by itself or in combination with another term, means, unless otherwise stated, a stable straight or branched chain hydrocarbon, or combinations thereof, fully saturated or containing from 1 to 3 degrees of unsaturation, consisting of the stated number of carbon atoms and from one to ten, preferably one to three, heteroatoms selected from the group consisting of O, N, Si and S, and wherein the nitrogen and sulfur atoms may optionally be oxidized and the nitrogen heteroatom may optionally be quaternized. The heteroatom (s) O, N and S may be placed at any interior position of the heteroalkyl group or at the position at which the alkyl group is attached to the remainder of the molecule. The heteroatom Si may be placed at any position of the heteroalkyl group, including the position at which the alkyl group is attached to the remainder of the molecule. Examples include —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —NH—CH₂—CH₂—NH—C(O)—CH₂—CH₃, —CH₂—CH₂—S(O)₂—CH₃, —CH═CH—O—CH₃, —Si(CH₃)₃, —CH₂—CH═N—O—CH₃, and —CH═CH—N(CH₃)—CH₃. Up to two heteroatoms may be consecutive, such as, for example, —CH₂—NH—OCH₃ and —CH₂—O—Si(CH₃)₃. Typically, a C₁ to C₄ heteroalkyl or heteroalkylene has 1 to 4 carbon atoms and 1 or 2 heteroatoms and a C₁ to C₃ heteroalkyl or heteroalkylene has 1 to 3 carbon atoms and 1 or 2 heteroatoms. In some aspects, a heteroalkyl or heteroalkylene is saturated.

Unless otherwise indicated, the term “heteroalkylene” by itself or in combination with another term means a divalent group derived from heteroalkyl (as discussed above), as exemplified by —CH₂—CH₂—S—CH₂—CH₂— and —CH₂—S—CH₂—CH₂—NH—CH₂—. For heteroalkylene groups, heteroatoms can also occupy either or both of the chain termini. Still further, for alkylene and heteroalkylene linking groups, no orientation of the linking group is implied.

“Protecting group” as used here means a moiety that prevents or reduces the ability of the atom or functional group to which it is linked from participating in unwanted reactions. Typical protecting groups for atoms or functional groups are given in Greene (1999), “PROTECTIVE GROUPS IN ORGANIC SYNTHESIS, 3^{RD} ED.”, WILEY INTERSCIENCE. Protecting groups for heteroatoms such as oxygen, sulfur and nitrogen are used in some instances to minimize or avoid unwanted their reactions with electrophilic compounds. In other instances, the protecting group is used to reduce or eliminate the nucleophilicity and/or basicity of the unprotected heteroatom. Non-limiting examples of protected oxygen are given by OR^{PR}, wherein R^{PR} is a protecting group for hydroxyl, wherein hydroxyl is typically protected as an ester (e.g. acetate, propionate or benzoate). Other protecting groups for hydroxyl avoid interfering with the nucleophilicity of organometahic reagents or other highly basic reagents, where hydroxyl is typically protected as an ether, including alkyl or heterocycloalkyl ethers, (e.g., methyl or tetrahydropyranyl ethers), alkoxymethyl ethers (e.g., methoxymethyl or ethoxymethyl ethers), optionally substituted aryl ethers, and silyl ethers (e.g., trimethylsilyl (TMS), triethylsilyl (TES), tert-butyldiphenylsilyl (TBDPS), tert-butyldimethylsilyl (TBS/TBDMS), triisopropylsilyl (TIPS) and [2-(trimethylsilyl)ethoxy]-methylsilyl (SEM)). Nitrogen protecting groups include those for primary or secondary amines as in —NHR^{PR} or —N(R^{PR})₂—, wherein least one of R^{PR} is a nitrogen atom protecting group or both R^{PR} together comprise a protecting group.

A protecting group is suitable when it is capable of preventing or avoiding unwanted side-reactions or premature loss of the protecting group under reaction conditions required to effect desired chemical transformation elsewhere in the molecule and during purification of the newly formed molecule when desired, and can be removed under conditions that do not adversely affect the structure or stereochemical integrity of that newly formed molecule. By way of example and not limitation, a suitable protecting group may include those previously described for protecting functional groups. A suitable protecting group is sometimes a protecting group used in peptide coupling reactions.

As used herein, the term “pharmaceutically acceptable salt” refers to organic or inorganic salts of a compound of the present disclosure that have specified toxicity and/or biodistribution properties. Suitable salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, and/or pamoate (i.e., 1,1′-methylene-bis-(2-hydroxy-3-naphthoate)) salts. The pharmaceutically acceptable salt may balance charge on the parent compound by being present as a counterion. More than one counterion may be present. When multiple counterions are present, the compounds may be present as a mixed pharmaceutically acceptable salt.

Pharmaceutically acceptable salts and/or hydrates of the mitofusin activators may also be present in the compositions of the present disclosure. As used herein, the term “pharmaceutically acceptable solvate” refers to an association between one or more solvent molecules and a mitofusin activator of the present disclosure or a salt thereof, wherein the solvate has specified toxicity and/or biodistribution properties. Examples of solvents that may form pharmaceutically acceptable solvates include, but are not limited to, water, isopropanol, ethanol, methanol, DMSO, ethyl acetate, acetic acid, and/or ethanolamine. As used herein, the term “pharmaceutically acceptable hydrate” refers to a mitofusin activator of the present disclosure or a salt thereof that further includes a stoichiometric or non-stoichiometric amount of water bound by non-covalent intermolecular forces, wherein the hydrate has specified toxicity and/or biodistribution properties.

The mitofusin activators described herein may be formulated using one or more pharmaceutically acceptable excipients (carriers) known to persons having ordinary skill in the art. The term “pharmaceutically acceptable excipient,” as used herein, refers to substances or components that do not cause unacceptable losses of pharmacological activity or unacceptable adverse side effects when administered to a subject. Example “pharmaceutically acceptable excipients” include, but are not limited to, solvents, dispersion media, coatings, antibacterial agents, antifungal agents, isotonic, and absorption delaying agents, provided that any of these agents do not produce significant side effects or are incompatible with the mitofusin activator in the composition. Example excipients are described, for example, in Remington's Pharmaceutical Sciences (A. R. Gennaro, Ed.), 21st edition, ISBN: 0781746736 (2005) and United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF may also be used. Such formulations may contain a therapeutically effective amount of one or more mitofusin activators, optionally as a salt, hydrate, and/or solvate, together with a suitable amount of excipient to provide a form for proper administration to a subject.

Compositions of the present disclosure may be stable to specified storage conditions. A “stable” composition refers to a composition having sufficient stability to allow storage at a convenient temperature, such as from about 0° C. to about 60° C. or about −20° C. to about 50° C., for a commercially reasonable period of time, such as at least about one day, at least about one week, at least about one month, at least about three months, at least about six months, at least about one year, or at least about two years.

Compositions of the present disclosure may be tailored to suit a desired mode of administration, which may include, but are not limited to, parenteral, pulmonary, oral, topical, transdermal, intradermal, intramuscular, intraperitoneal, intravenous, subcutaneous, intranasal, epidural, ophthalmic, pulmonary, epidural, buccal, and rectal. The compositions may also be administered in combination with one or more additional agents or together with other biologically active or biologically inert agents.

Controlled-release (or sustained-release) compositions may be formulated to extend the activity of the mitofusin activators and reduce dosing frequency. Controlled-release compositions may also be used to affect the time of onset of action or other characteristics, such as plasma levels of the mitofusin activator, and consequently affect the occurrence of side effects. Controlled-release compositions may be designed to initially release an amount of one or more mitofusin activators that produces the desired therapeutic effect, and gradually and continually release other amounts of the mitofusin activator to maintain the level of therapeutic effect over an extended period. In order to maintain a near-constant level of mitofusin activator in the body, the mitofusin activator may be released at a rate sufficient to replace the amount being metabolized or excreted from a subject. The controlled-release may be stimulated by various inducers (e.g., change in pH, change in temperature, enzymes, water, or other physiological conditions or molecules).

Agents or compositions described herein may also be used in combination with other therapeutic modalities, as described further below. Thus, in addition to the therapies described herein, one may also provide to the subject other therapies known to be efficacious for treatment of a disease, disorder, or condition being targeted by the mitofusin activator or a related disease, disorder, or condition.

Mitofusin activators of the present disclosure may stimulate mitochondrial fusion, increase mitochondrial fitness, and enhance mitochondrial subcellular transport. Accordingly, in another aspect of the present disclosure, any one or a combination of mitofusin activators of the present disclosure or a pharmaceutically acceptable salt thereof may be administered in a therapeutically effective amount to a subject having or suspected of having a mitochondria-associated disease, disorder or condition. The subject may be a human or other mammal having or suspected of having a mitochondria-associated disease, disorder or condition.

The mitochondria-associated disease, disorder or condition may be a pheripheral nervous system (PNS) or central nervous system (CNS) genetic or non-genetic disorder, physical damage, and/or chemical injury. In some aspects, in the method of treating a disease, disorder or condition for which a mitofusin activator is indicated, the PNS or CNS disorder may be selected from any one or a combination of: a chronic neurodegenerative condition wherein mitochondrial fusion, fitness, or trafficking are impaired; a disease or disorder associated with mitofusin-1 (MFN1) or mitofusin-2 (MFN2) dysfunction; a disease associated with mitochondrial fragmentation, dysfunction, or dysmotility; a degenerative neuromuscular condition such as Charcot-Marie-Tooth disease, amyotrophic lateral sclerosis (ALS), Huntington's disease, Alzheimer's disease, Parkinson's disease, hereditary motor and sensory neuropathy, autism, autosomal dominant optic atrophy (ADOA), muscular dystrophy, Lou Gehrig's disease, cancer, mitochondrial myopathy, diabetes mellitus and deafness (DAD), Leber's hereditary optic neuropathy (LHON), Leigh syndrome, subacute sclerosing encephalopathy, neuropathy, ataxia, retinitis pigmentosa, and ptosis (NARP), myoneurogenic gastrointestinal encephalopathy (MNGIE), myoclonic epilepsy with ragged red fibers (MERRF), mitochondrial myopathy, encephalomyopathy, lactic acidosis, stroke-like symptoms (MELAS), ntDNA depletion, mitochondrial neurogastrointestinal encephalomyopathy (MNGIE), dysautonomic mitochondrial myopathy, mitochondrial channelopathy, or pyruvate dehydrogenase complex deficiency (PDCD/PDIHI), diabetic neuropathy, chemotherapy-induced peripheral neuropathy, crush injury, SCI, traumatic brain injury (TBI), stroke, optic nerve injury, and/or related conditions that involve axonal disconnection.

Other mitochondria-associated diseases, disorders, or conditions that may be treated with the compositions disclosed herein, but are not limited to, Alzheimer's disease, ALS, Alexander disease, Alpers' disease, Alpers-Huttenlocher syndrome, alpha-methylacyl-CoA racemase deficiency, Andermann syndrome, Arts syndrome, ataxia neuropathy spectrum, ataxia (e.g., with oculomotor apraxia, autosomal dominant cerebellar ataxia, deafness, and narcolepsy), autosomal recessive spastic ataxia of Charlevoix-Saguenay, Batten disease, beta-propeller protein-associated neurodegeneration, cerebro-oculo-facio-skeletal syndrome (COFS), corticobasal degeneration CLN1 disease, CLN10 disease, CLN2 disease, CLN3 disease, CLN4 disease, CLN6 disease, CLN7 disease, CLN8 disease, cognitive dysfunction, congenital insensitivity to pain with anhidrosis, dementia, familial encephalopathy with neuroserpin inclusion bodies, familial British dementia, familial Danish dementia, fatty acid hydroxylase-associated neurodegeneration, Friedreich's Ataxia, Gerstmann-Straussler-Scheinker Disease, GM2-gangliosidosis (e.g., AB variant), HMSN type 7 (e.g., with retinitis pigmentosa), Huntington's disease, infantile neuroaxonal dystrophy, infantile-onset ascending hereditary spastic paralysis, infantile-onset spinocerebellar ataxia, juvenile primary lateral sclerosis, Kennedy's disease, Kuru, Leigh's Disease, Marinesco-Sjögren syndrome, mild cognitive impairment (MCI), mitochondrial membrane protein-associated neurodegeneration, motor neuron disease, monomelic amyotrophy, motor neuron diseases (MND), multiple system atrophy, multiple system atrophy with orthostatic hypotension (Shy-Drager Syndrome), multiple sclerosis, multiple system atrophy, neurodegeneration in down's syndrome (NDS), neurodegeneration of aging, neurodegeneration with brain iron accumulation, neuromyelitis optica, pantothenate kinase-associated neurodegeneration, opsoclonus myoclonus, prion disease, progressive multifocal leukoencephalopathy, Parkinson's disease, Parkinson's disease-related disorders, polycystic lipomembranous osteodysplasia with sclerosing leukoencephalopathy, prion disease, progressive external ophthalmoplegia, riboflavin transporter deficiency neuronopathy, Sandhoff disease, spinal muscular atrophy (SMA), spinocerebellar ataxia (SCA), striatonigral degeneration, transmissible spongiform encephalopathies (prion diseases), and/or Wallerian-like degeneration.

Still other mitochondria-associated diseases, disorders, or conditions that may be treated with the compositions disclosed herein include abulia; agraphia; alcoholism; alexia; alien hand syndrome; Allan-Herndon-Dudley syndrome; alternating hemiplegia of childhood; Alzheimer's disease; amaurosis fugax; amnesia; LS; aneurysm; angelman syndrome; anosognosia; aphasia: apraxia; arachnoiditis; Arnold-Chiari malformation; asomatognosia; Asperger syndrome; ataxia; attention deficit hyperactivity disorder; atr-16 syndrome; auditory processing disorder; autism spectrum; Behcets disease; bipolar disorder; Bell's palsy; brachial plexus injury; brain damage; brain injury; brain tumor; Brody myopathy; Canavan disease; capgras delusion; carpal tunnel syndrome; causalgia; central pain syndrome; central pontine myelinolysis; centronuclear myopathy; cephalic disorder; cerebral aneurysm; cerebral arteriosclerosis; cerebral atrophy; cerebral autosomal dominant arteriopathy with subcortical infarcts and leukoencephalopathy (CADASIL); cerebral dysgenesis-neuropathy-ichthyosis-keratoderma syndrome (CEDNIK syndrome); cerebral gigantism; cerebral palsy; cerebral vasculitis; cervical spinal stenosis; Charcot-Marie-Tooth disease; chiari malformation; chorea; chronic fatigue syndrome; chronic inflammatory demyelinating polyneuropathy (CIDP); chronic pain; Cockayne syndrome; Coffin-Lowry syndrome; coma; complex regional pain syndrome; compression neuropathy; congenital facial diplegia; corticobasal degeneration; cranial arteritis; craniosynostosis; Creutzfeldt-Jakob disease; cumulative trauma disorders: Cushing's syndrome; cyclothymic disorder; cyclic vomiting syndrome (CVS); cytomegalic inclusion body disease (CIBD); cytomegalovirus infection; Dandy-Walker syndrome; dawson disease; de Morsier's syndrome; Dejerine-Klumpke palsy; Dejerine-Sottas disease; delayed sleep phase syndrome; dementia; dermatomyositis; developmental coordination disorder; diabetic neuropathy; diffuse sclerosis; diplopia; disorders of consciousness; down syndrome; Dravet syndrome; duchenne muscular dystrophy; dysarthria; dysautonomia; dyscalculia; dysgraphia; dyskinesia; dyslexia; dystonia; empty sella syndrome; encephalitis; encephalocele; encephalotrigeminal angiomatosis; encopresis; enuresis; epilepsy; epilepsy-intellectual disability in females; erb's palsy; erythromelalgia; essential tremor; exploding head syndrome; Fabry's disease; Fahr's syndrome; fainting; familial spastic paralysis; febrile seizures: Fisher syndrome; Friedreich's ataxia; fibromyalgia; Foville's syndrome, fetal alcohol syndrome; fragile x syndrome; fragile x-associated tremor/ataxia syndrome (FXTAS); Gaucher's disease; generalized epilepsy with febrile seizures plus; Gerstmann's syndrome; giant cell arteritis; giant cell inclusion disease; globoid cell leukodystrophy; gray matter heterotopia; Guillain-Barré syndrome; generalized anxiety disorder; HTLV-1 associated myelopathy; Hallervorden-Spatz syndrome; head injury; headache; hemifacial spasm; hereditary spastic paraplegia; heredopathia atactica polyneuritiformis; herpes zoster oticus; herpes zoster; Hirayama syndrome; Hirschsprung's disease; Holmes-Adie syndrome; holoprosencephaly; Huntington's disease; hydranencephaly; hydrocephalus; hypercortisolism; hypoxia; immune-mediated encephalomyelitis; inclusion body myositis; incontinentia pigmenti; infantile refsum disease; infantile spasms; inflammatory myopathy; intracranial cyst; intracranial hypertension: isodicentric 15; Joubert syndrome; Karak syndrome; Kearns-Sayre syndrome; Kinsbourne syndrome; Kleine-Levin syndrome; Klippel Feil syndrome; Krabbe disease; Kufor-Rakeb syndrome; Lafora disease; Lambert-Eaton myasthenic syndrome; Landau-Kleffner syndrome; lateral medullary (Wallenberg) syndrome; learning disabilities; Leigh's disease; Lennox-Gastaut syndrome; Lesch-Nyhan syndrome; leukodystrophy; leukoencephalopathy with vanishing white matter; lewy body dementia; lissencephaly; locked-in syndrome; Lou Gehrig's disease (amyotrophic lateral sclerosis (ALS)); lumbar disc disease: lumbar spinal stenosis; lyme disease—neurological sequelae; Machado-Joseph disease (spinocerebellar ataxia type 3); macrencephaly; macropsia; mal de debarquement; megalencephalic leukoencephalopathy with subcortical cysts; megalencephaly; Melkersson-Rosenthal syndrome; menieres disease; meningitis; Menkes disease; metachromatic leukodystrophy; microcephaly; micropsia; migraine; Miller Fisher syndrome; mini-stroke (transient ischemic attack); misophonia; mitochondrial niyopathy; mobius syndrome; monomelic amyotrophy; Morvan syndrome; motor neurone disease—see ALS; motor skills disorder; moyamoya disease; mucopolysaccharidoses; multi-infarct dementia; multifocal motor neuropathy; multiple sclerosis; multiple system atrophy; muscular dystrophy; myalgic encephalomyelitis; myasthenia gravis; myelinoclastic diffuse sclerosis; myoclonic encephalopathy of infants; myoclonus; myopathy; myotubular myopathy; myotonia congenita; narcolepsy; neuro-Behçet's disease; neurofibromatosis; neuroleptic malignant syndrome; neurological manifestations of aids; neurological sequelae of lupus; neuromyotonia; neuronal ceroid lipofuscinosis; neuronal migration disorders; neuropathy; neurosis; Niemann-Pick disease; non-24-hour sleep-wake disorder; nonverbal learning disorder; O'Sullivan-McLeod syndrome; occipital neuralgia; occult spinal dysraphism sequence; Obtahara syndrome; olivopontocerebellar atrophy; opsoclonus myoclonus syndrome; optic neuritis; orthostatic hypotension; otosclerosis; overuse syndrome; palinopsia; paresthesia; Parkinson's disease; paramyotonia congenita; paraneoplastic diseases; paroxysmal attacks; Parry-Romberg syndrome; pediatric autoimmune neuropsychiatric disorders associated with streptococcal infections (PANDAS); Pelizaeus-Merzbacher disease; periodic paralyses; peripheral neuropathy; pervasive developmental disorders; phantom limb/phantom pain; photic sneeze reflex; phytanic acid storage disease; Pick's disease; pinched nerve; pituitary tumors; ping; polyneuropathy; polio; polymicrogyria; polymyositis; porencephaly; post-polio syndrome; postherpetic neuralgia (phn); postural hypotension; Prader-Willi syndrome; primary lateral sclerosis; prion diseases; progressive hemifacial atrophy; progressive multifocal leukoencephalopathy; progressive supranuclear palsy; prosopagnosia; pseudotumor cerebri; quadrantanopia; quadriplegia; rabies; radiculopathy; Ramsay Hunt syndrome type 1; Ramsay Hunt syndrome type 2; Ramsay Hunt syndrome type 3—see Ramsay-Hunt syndrome; Rasmussen encephalitis; reflex neurovascular dystrophy; refsum disease; REM sleep behavior disorder; repetitive stress injury; restless legs syndrome; retrovirus-associated myelopathy; Rett syndrome; Reye's syndrome; rhythmic movement disorder; Romberg syndrome; Saint Vitus' dance; Sandhoff disease; Schilder's disease (two distinct conditions); schizencephaly; sensory processing disorder; septo-optic dysplasia; shaken baby syndrome; shingles; Shy-Drager syndrome; Sjögren's syndrome; sleep apnea; sleeping sickness; snatiation; Sotos syndrome; spasticity; spina bifida; spinal cord injury; spinal cord tumors; spinal muscular atrophy; spinal and bulbar muscular atrophy; spinocerebellar ataxia; split-brain; Steele-Richardson-Olszewski syndrome; stiff-person syndrome; stroke; Sturge-Weber syndrome; stuttering; subacute sclerosing panencephalitis; subcortical arteriosclerotic encephalopathy; superficial siderosis; Sydenham's chorea; syncope; synesthesia; syringomyelia; tarsal tunnel syndrome; tardive dyskinesia; tardive dysphrenia; Tarlov cyst; Tay-Sachs disease; temporal arteritis; temporal lobe epilepsy; tetanus; tethered spinal cord syndrome; Thomsen disease; thoracic outlet syndrome; tic douloureux; Todd's Paralysis; tourette syndrome; toxic encephalopathy; transient ischemic attack; transmissible spongiform encephalopathies; transverse myelitis; traumatic brain injury; tremor; trichotillomania; trigeminal neuralgia; tropical spastic paraparesis; trypanosomiasis; tuberous sclerosis; 22q13 deletion syndrome; Unverricht-Lundborg disease; vestibular schwannoma (acoustic neuroma); Von Hippel-Lindau disease (VHL); viliuisk encephalomyelitis (VE); Wallenberg's syndrome; west syndrome; whiplash; Williams syndrome; Wilson's disease; y-linked hearing impairment; and/or Zellweger syndrome.

Each of the states, diseases, disorders, and conditions, described herein, as well as others, can benefit from compositions and methods described herein. Generally, treating a state, disease, disorder, or condition includes preventing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition (e.g., arresting or reducing the development of the disease or at least one clinical or subclinical symptom thereof). Furthermore, treating can include relieving the disease (e.g., causing regression of the state, disease, disorder, or condition or at least one of its clinical or subclinical symptoms). A benefit to a subject to be treated can be either statistically significant or at least perceptible to the subject or to a physician.

A mitochondria-associated disease, disorder, or condition may be a disease primarily caused by or secondarily associated with mitochondrial dysfunction, fragmentation, or loss-of-fusion, or associated with dysfunction in MFN1 or MFN2 catalytic activity or conformational unfolding. Mitochondrial dysfunction may be caused by genetic mutations of mitofusins or other (nuclear or mitochondrial encoded) genes, or may be caused by physical, chemical, or environmental injury to the CNS or PNS.

In a particular example, cancer chemotherapy-induced sensory and motor neuropathies may be prevented or treated with the compositions of the present disclosure. Chemotherapy-induced peripheral neuropathy is one of the most common complications of cancer chemotherapy, affecting 20% of all patients and almost 100% of patients receiving high doses of chemotherapeutic agents. Dose-dependent neurotoxicity of motor and sensory neurons can lead to chronic pain, hypersensitivity to hot, cold, and mechanical stimuli, and/or impaired neuromuscular control. The most common chemotherapeutic agents linked to CIPN are platinum, vinca alkaloids, taxanes, epothilones, and the targeted proteasome inhibitor, bortezomib.

CIPN most commonly affects peripheral sensory neurons whose cell bodies are located in dorsal root ganglia lacking the blood-brain barrier that protects other components of the central and peripheral nervous system. Unprotected dorsal root ganglion neurons are more sensitive to neuronal hyperexcitability and innate immune system activation evoked by circulating cytotoxic chemotherapeutic agents. CIPN affects quality of life, and is potentially disabling, because it provokes chronic neuropathic pain that, like other causes of neuralgia (e.g., post herpetic neuralgia, diabetic mononeuropathy), is refractory to analgesic therapy. Motor nerve involvement commonly manifests as loss of fine motor function with deterioration in hand writing, difficulty in buttoning clothes or sewing, and sometimes upper and lower extremity weakness or loss of endurance. CIPN typically manifests within weeks of chemotherapy and in many cases improves after chemotherapy treatment ends, although residual pain, sensory, or motor defects are observed in one-third to one-half of affected patients. Unfortunately, CIPN-limited chemotherapy dosing can lead to delays, reduction, or interruption of cancer treatment, thus shortening survival.

Mitochondrial dysfunction and oxidative stress are implicated in CIPN because of observed ultrastructural morphological abnormalities, impaired mitochondria DNA transcription and replication, induction of mitochondrial apoptosis pathways, and reduction of experimental CIPN signs by anticipatory mitochondrial protection. Mitofusin activators may enhance overall mitochondrial function in damaged neurons, increase mitochondrial transport to areas of neuronal damage, and accelerate in vitro neuron repair/regeneration after chemotherapy-induced damage. For this reason, it is believed that mitofusin activators may reduce neuronal injury conferred by chemotherapeutic agents in CIPN and accelerate regeneration/repair of nerves damaged by chemotherapeutic anticancer agents. As such, the present disclosure provides for compositions and methods to treat cancer chemotherapy induced nerve injury and neuropathy.

In another example, injury in the CNS or PNS (e.g., trauma to the CNS or PNS, crush injury, SCI, TBI, stroke, optic nerve injury, or related conditions that involve axonal disconnection) may be treated with the compositions of the present disclosure. The CNS includes the brain and the spinal cord and the PNS is composed of cranial, spinal, and autonomic nerves that connect to the CNS

Damage to the nervous system caused by mechanical, thermal, chemical, or ischemic factors may impair various nervous system functions such as memory, cognition, language, and voluntary movement. Most often, this is through accidental crush or transection of nerve tracts, or as an unintended consequence of medical interventions, that interrupt normal communications between nerve cell bodies and their targets. Other types of injuries may include disruption of the interrelations between neurons and their supporting cells or the destruction of the blood-brain barrier.

Mitofusin activators may rapidly reverse mitochondrial dysmotility in neurons from mice or patients with various genetic or chemotherapeutic neurodegenerative diseases, in axons injured by chemotherapeutic agents, and in axons severed by physical injury. For this reason, mitofusin activators may enhance regeneration/repair of physically damaged nerves, as in vehicular and sports injuries, penetration trauma from military or criminal actions, and iatrogenic injury during invasive medical procedures. As such, the present disclosure provides for compositions and methods to treat physical nerve injury.

Mitochondrial motility is also implicated in neuropathy and traumatic crush or severance nerve injuries. After nerve laceration or crush injury, nerves will either regenerate and restore neuromuscular function or fail to regenerate such that neuromuscular function in permanently impaired. Mitofusin activators may increase mitochondrial trafficking, thereby enabling a nerve to regenerate after traumatic injuries.

The amount of a mitofusin activator and excipient to produce a composition in a given dosage form may vary depending upon the subject being treated, the condition being treated and the particular mode of administration. It will be appreciated that the unit content of mitofusin activator contained in an individual dose of a given dosage form need not in itself constitute a therapeutically effective amount, as the necessary therapeutically effective amount could be reached by administration of a number of individual doses, or the therapeutic effect may be cumulative over time.

Dosing of the mitofusin activators of the present disclosure may occur as a single event or over a time course of treatment. For example, a mitofusin activator may be administered daily, weekly, bi-weekly, or monthly. For treatment of acute conditions, the time course of treatment may be at least several days, with dosing taking place at least once a day or continuously. Certain conditions could extend treatment from several days to several weeks. For example, treatment could extend over one week, two weeks, or three weeks. For chronic conditions, treatment could extend from several weeks to several months or even years.

Toxicity and therapeutic efficacy of the compositions described herein may be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that may be expressed as the ratio LD₅₀/ED₅₀, where larger therapeutic indices are generally understood in the art to be optimal.

Unless indicated to the contrary, the numerical parameters set forth in the following specification and attached claims are approximations that may vary depending upon the desired properties sought to be obtained by the embodiments of the present invention. At the very least, and not as an attempt to limit the application of the doctrine of equivalents to the scope of the claim, each numerical parameter should at least be construed in light of the number of reported significant digits and by applying ordinary rounding techniques.

One or more illustrative embodiments incorporating various features are presented herein. Not all features of a physical implementation are described or shown in this application for the sake of clarity. It is understood that in the development of a physical embodiment incorporating the embodiments of the present invention, numerous implementation-specific decisions must be made to achieve the developer's goals, such as compliance with system-related, business-related, government-related and other constraints, which vary by implementation and from time to time. While a developer's efforts might be time-consuming, such efforts would be, nevertheless, a routine undertaking for those of ordinary skill in the art and having benefit of this disclosure.

While various systems, tools and methods are described herein in terms of “comprising” various components or steps, the systems, tools and methods can also “consist essentially of” or “consist of” the various components and steps.

As used herein, the phrase “at least one of” preceding a series of items, with the terms “and” or “or” to separate any of the items, modifies the list as a whole, rather than each member of the list (i.e., each item). The phrase “at least one of” allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Therefore, the disclosed systems, tools and methods are well adapted to attain the ends and advantages mentioned as well as those that are inherent therein. The particular embodiments disclosed above are illustrative only, as the teachings of the present disclosure may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope of the present disclosure. The systems, tools and methods illustratively disclosed herein may suitably be practiced in the absence of any element that is not specifically disclosed herein and/or any optional element disclosed herein. While systems, tools and methods are described in terms of “comprising,” “containing,” or “including” various components or steps, the systems, tools and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces. If there is any conflict in the usages of a word or term in this specification and one or more patent or other documents that may be incorporated herein by reference, the definitions that are consistent with this specification should be adopted.

### SYNTHESIS OF THE REFERENCE COMPOUNDS

Reference compounds were used for comparison with the compounds of the present disclosure. The reference compounds were synthesized following previously described procedures.

### Synthesis of (N-(trans-4-hydroxycyclohexyl)-5-phenylpentanamide (Compound 1)

This mitofusin activator was prepared as described in U.S. Patent Application Publication 2020/0345668, which is incorporated herein by reference.

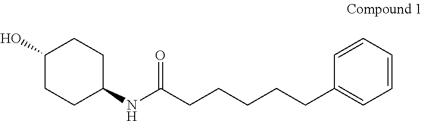 [SMILES: O=C(CCCCCc1ccccc1)N[C@H]1CC[C@H](O)CC1]

Scheme 1-a below outlines the synthesis of Compound 1.

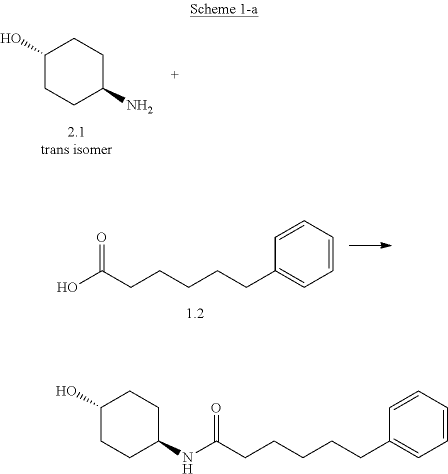 [SMILES: N[C@H]1CC[C@H](O)CC1 ; O=C(O)CCCCCc1ccccc1 ; O=C(CCCCCc1ccccc1)N[C@H]1CC[C@H](O)CC1]

To a solution of 6-phenylhexanoic acid 1.2 (200 mg. 1.04 mmol, 196 μL, 1.00 eq.) and trans-4-hydroxycyclohexylamine 2.1 (174 mg, 1.14 mmol, 1.10 eq.) and DIEA (269 mg, 2.08 mmol, 362 μL, 2.00 eq.) in DMF (3 mL) was added HOBt (169 mg, 1.25 mmol, 1.20 eq.) and EDC1 (299 mg, 1.56 mmol, 1.50 eq.). The mixture was stirred at 10° C. for 10 hours. The mixture was diluted with water (20 mL) and extracted with EtOAC (10 mL×3). The organic phase was washed with 1M aqueous HC1 (30 mL), dried over Na₂SO₄, filtered and concentrated under reduced pressure to give a residue. The residue was purified by preparative HPLC. The title compound was obtained as a white solid. MS: m/z=290.1 (M+H)⁺; ¹H NMR (400 MHz): MeOD δ 7.25-7.21 (m, 2H), 7.16-7.11 (m, 3H), 3.59-3.48 (m, 1H), 2.60 (t, J=7.6 Hz, 2H), 2.13 (t, J=7.6 Hz, 2H), 2.11-1.92 (m, 2H), 1.92-1.83 (m, 2H), 1.65-1.60 (m, 4H), 1.34-1.19 (m, 6H). ¹³C NMR (400 MHz): MeOD δ 175.660, 143.882, 129.590, 129.441, 126.819, 70.591, 37.231, 36.864, 34.965, 32.519, 31.655, 29.804, 27.098.

Mitofusin activators having structures similar to those of Compound 1 are represented by Compound 23-27 below and were also synthesized by a similar method.

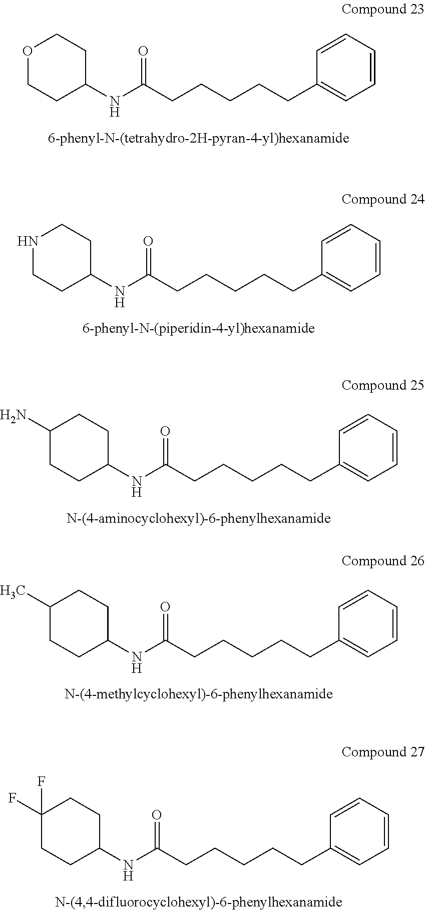 [SMILES: O=C(CCCCCc1ccccc1)NC1CCOCC1 ; O=C(CCCCCc1ccccc1)NC1CCNCC1 ; NC1CCC(NC(=O)CCCCCc2ccccc2)CC1 ; CC1CCC(NC(=O)CCCCCc2ccccc2)CC1 ; O=C(CCCCCc1ccccc1)NC1CCC(F)(F)CC1]

### Synthesis of N-((1r,4r)-4-hydroxycyclohexyl)-2-(3-phenylpropyl)cyclopropane-1-carboxamide (Compound 2)

Scheme 1-b below outlines the synthesis of N-((1r,4r)-4-hydroxycyclohexyl)-2-(3-phenylpropyl)cyclopropane-1-carboxamide (Compound 2).

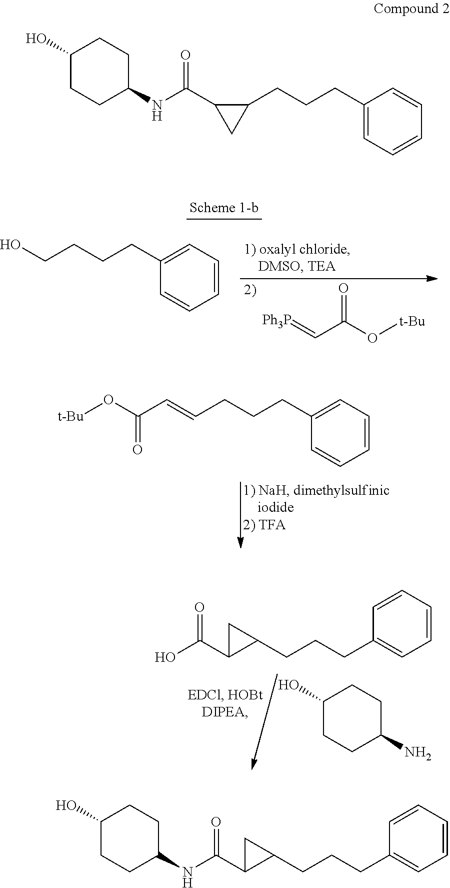

To a solution of oxalyl chloride (4.65 g, 36.6 mmol, 3.20 ml, 110 eq) in DCM (75.0 mL) cooled to −55° C. under N₂ atmosphere, a solution of DMSO (5.72 g, 73.2 mmol, 5.72 mL, 2.20 eq) in DCM (30.0 mL) was dropwise. After stirring for 5 min, 4-phenylbutan-1-ol was added dropwise (5.00 g, 33.2 mmol, 5.08 mL, 1.00 eq) in DCM (15.0 mL). After stirring for 15 min, TEA (16.8 g, 166 mmol, 23.1 mL, 5.00 eq) was added, and the reaction mixture was warmed to 25° C. To the warmed reaction mixture was then added 100 mL 1 N HCl, and the product was extracted with DCM 200 mL (100 mL×2). The combined organic layers were washed with water 50 ml, dried over Na₂SO₄, filtered and concentrated to give 4-phenylbutanal (5.00 g).

To a solution of 4-phenylbutanal (5.00 g, 33.7 mmol, 9.80 mL, 1.00 eq) in THF (50.0 mL) was added tert-butyl 2-(triphenyl-5-phosphanylidene)acetate (16.5 g, 43.8 mmol, 1.30 eq). The reaction mixture was stirred at 20° C. for 12 hrs to give tert-butyl (E)-6-phenylhex-2-enoate (6.00 g, 24.3 mmol, 72.1% yield).

To a suspension of NaH (1.17 g, 29.2 mmol, 60.0% purity, 1.20 eq) in DMSO (30.0 mL) was added dimethylmethanesulfinic iodide (6.43 g, 29.2 mmol, 1.20 eq). The mixture was stirred at 20° C. for 0.5 hr, and tert-butyl (E)-6-phenylhex-2-enoate (6.00 g, 24.3 mmol, 1.00 eq) in DMSO (3.00 mL) was added. The reaction mixture was stirred at 20° C. for 1 hr to give tert-butyl 2-(3-phenylpropyl)cyclopropane-1-carboxylate (2.10 g, 8.07 mmol, 33.1% yield). Removal of the t-butyl ester was accomplished by adding TFA TFA (7.70 g, 67.5 mmol, 5.00 mL, 17.5 eq) to a solution of tert-butyl 2-(3-phenylpropyl)cyclopropane-1-carboxylate (1.00 g, 3.84 mmol, 1.00 eq) in DCM (5.00 mL). After stirring at 25° C. for 15 hrs, 2-(3-phenylpropyl)cyclopropane-1-carboxylic acid (800 mg) was obtained.

EDCI (1.00 g, 5.22 mmol, 1.50 eq), HOBt (564 ng, 4.18 mmol, 1.20 eq), DIPEA (1.35 g, 10.4 mmol, 1.82 mL, 3.00 eq), and trans-4-aminocyclohexan-1-ol (580 mg, 3.83 mmol, 1.10 eq, HCl) were added to a solution of 2-(3-phenylpropyl)cyclopropane-1-carboxylate (800 mg, 3.48 mmol, 1.00 eq) in DMF (8.00 mL) and stirred at 25° C. for 16 hrs. After solvent removal, the residue was purified by preparative HPLC (column: Waters Xbridge C18 150*50 mm*10 μm; mobile phase: [water (10 mM NH₄HCO₃)-ACN]; B %: 28%-58%, 11.5 min) to give the title compound as a white solid. LC-MS: Rₜ=0.904 min, m/z=302.1 (M+H)⁺. HPLC: Rt=2.898 min, purity: 98.6%, tinder 220 nm. ¹³C NMR: (400 MHz, MeOD) δ 173.97, 142.27, 127.96, 127.89, 125.31, 69.07, 35.14, 33.45, 32.23, 30.87, 3024, 21.27, 20.55, 13.04. ¹H NMR: (400 MHz, MeOD) δ 7.27-7.24 (m, 2H), 7.18-7.15 (m, 3H), 3.64-3.61 (m, 1H), 3.55-3.50 (m, 1H), 2.64 (t, J=8 Hz, 2H), 1.97-1.89 (m, 4H), 1.75-1.73 (m, 2H), 1.36-1.04 (m, 8H), 1.29-1.27 (m, 1H), 0.59-0.57 (m, 1H).

### Biological Assays

Compounds designed, selected and/or optimized by methods described above, once produced, can be characterized using a variety of assays known to those skilled in the art to determine whether the compounds have biological activity. For example, the molecules can be characterized by conventional assays, including but not limited to those assays described below, to determine whether they have a predicted activity, binding activity and/or binding specificity.

Furthermore, high-throughput screening can be used to speed up analysis using such assays. As a result, it can be possible to rapidly screen the molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below.

Various in vitro or in vivo biological assays may be suitable for detecting the effect of the compounds of the present disclosure. These in vitro or in vivo biological assays can include, but are not limited to, enzymatic activity assays, electrophoretic mobility shift assays, reporter gene assays, in vitro cell viability assays, and the assays described herein.

In some embodiments, the assay is an assay as described in U.S. Patent Application Publication Nos. 2020/0345668 and 2020/0345669 (incorporated herein by reference above).

In some embodiments, the biological assay involves evaluation of the dose-response of a compound of described herein, e.g., in Mfn1- or Mfn2-deficient cells.

In some embodiments, the biological assay involves evaluation of Mitofusin-stimulating activities of a compound of described herein, e.g., in Mfn1-null or Mfn2-null cells.

In some embodiments, the biological assay was performed with wild-type MEFs (e.g., prepared from E10.5 c57/bl6 mouse embryos).

In some embodiments, the biological assay was performed with SV-40 T antigen-immortalized MFN1 null (CRL-2992), MFN2 null (CRL-2993), and/or MFN1/MFN2 double null MEFs (CRL-2994).

In some embodiments, the biological assay involves evaluation of in vitro stability, e.g., in human and mouse liver microsomes.

In some embodiments, the biological assay involves parallel artificial membrane permeability assay (PAMPA)

In some embodiments, the PAMPA is performed with PVDF membrane, e.g., pre-coated with 5 μL of 1% brain polar lipid extract (porcine)/dodecane mixture.

### Exemplary Cell Lines

Wild-type MEFs were prepared from E10.5 c57/bl6 mouse embryos. SV-40 T antigen-immortalized MFN1 null (CRL-2992), MFN2 null (CRL-2993) and MFN1/MFN2 double null MEFs (CRL-2994) were purchased from ATCC. MEFs were subcultured in DMEM (4.5 g/L glucose) plus 10% fetal bovine serum, Ix nonessential amino acids, 2 mM L-glutamine, 100 units/mL penicillin and 100 μg/mL streptomycin.

### Exemplary Confocal Live Cell Studies of Mitochondria

Live cell imaging was performed on an Olympus Diaphot 200 fluorescence microscope equipped with a 60× water immersion objective. All live cells were grown on coated glass-bottom 12-well plates and studied in modified Krebs-Henseleit buffer (138 mM NaCl, 3.7 mM KCl, 1.2 mM KH₂PO₄ 15 mM, 20 mM HEPES and 1 mM CaCl₂))) at room temperature.

Cells were excited with 408 nm (Hoechst), 561 nm (MitoTracker Green and Calcein AM, GFP), or 637 nm (TMRE, MitoTracker Orange, Ethidium homodimer-1, and AF594-Dextran) laser diodes. For mitochondrial elongation studies, mitochondrial aspect ratio (long axis/short axis) was calculated using automated edge detection and Image J software. Mitochondrial depolarization was calculated as percent of green mitochondria visualized on MitoTracker Green and TMRE merged images, expressed as green/(green+yellow mitochondria)×100.

### Exemplary Embodiments

The present disclosure provides compositions comprising a mitofusin activator or a pharmaceutically acceptable salt thereof having a structure represented by Formula I

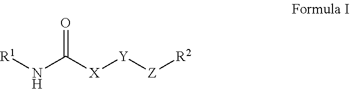

wherein R¹ is optionally substituted cycloalkyl or optionally substituted heterocycloalkyl, R² is an aryl or heteroaryl group, preferably optionally substituted phenyl, X is (CH₂)ₐ, Y is cyclopentyl or cyclohexyl, Z is (CH₂)_{b}, a is 0 or 1, and b is 1, 2 or 3. Collectively, X, Y and Z (and associated variables a and b) may be selected to form a 5- or 6-atom bridge between the amide carbonyl (C═O) and the phenyl ring (R²) in the mitofusin activator having a structure represented by Formula I. The 5- or 6-atom bridge refers to the shortest continuous chain of atoms extending between the amide carbonyl and the phenyl ring.

The cycloalkyl or heterocycloalkyl group R¹ in Formula I may be optionally substituted at any position by one or more of the following groups: amine, alkylamine, amide, alkylamide, alkoxy, aryloxy, hydroxyalkyl, azo, halo (F, Cl, Br, I), C₁₋₈ alkyl, carbonyl, carboxylic acids or carboxylic esters, cyano, C₃₋₈ cycloalkyl, C₃₋₈ heteroaryl, C₃₋₈ heterocyclyl, C₆-C₁₀ aryl, hydroxy, thiol, thioether, sulfoxide, sulfone, and sulfonamide, some of which may be optionally further substituted with acetamide, alkoxy, amino, azo, Br, (C₁₋₈ alkyl, carbonyl, carboxyl, Cl, cyano, C₃₋₈cycloalkyl, C₃₋₈ heteroaryl, C₃₋₈ heterocyclyl, hydroxyl, F, halo, indole, nitrile, phenyl, sulfoxide, sulfone, and/or thiophene. Heterocyclyl groups may contain one or more N, O, or S atoms within their ring structure.

In some examples, R¹ may be selected from among the following:

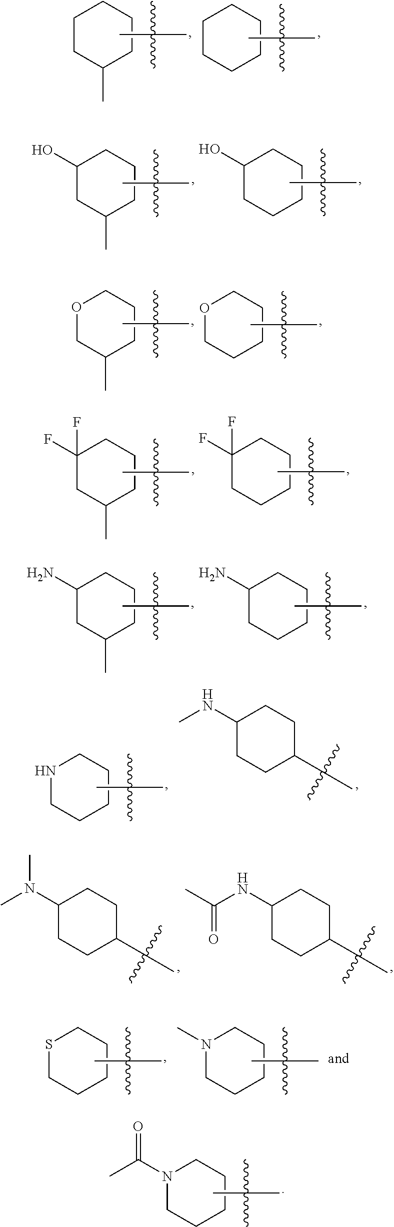

R² may be an optionally substituted phenyl group in Formula I. The phenyl group in Formula I may be optionally substituted by one or more of the following entities: amine, alkylamine, amide, alkylamide, alkoxy, aryloxy, hydroxyalkyl, azo, halo (F, C, Br, I), Cis alkyl, carbonyl, carboxylic acids or carboxylic esters, cyano, C₃₋₈ cycloalkyl, C₃₋₈ heteroaryl, C₃₋₈ heterocyclyl, C₆-C₁₀ aryl, hydroxy, thiol, thioether, sulfoxide, sulfone, and sulfonamide. The optional substitutions may be present at any available phenyl ring position. 0, 1, 2, 3, 4 or 5 optional substitutions may be present.

In the mitofusin activators disclosed herein, any regioisomer of the cyclopentyl group or the cyclohexyl group may be present. The cyclopentyl group may be 1,2-cyclopentyl or 1,3-cyclopentyl. The cyclohexyl group may be 1,2-cyclohexyl, 1,3-cyclohexyl, or 1,4-cyclohexyl. Such cyclopentyl or cyclohexyl groups may be directly bonded to the amide carbonyl group (a=0) or spaced apart therefrom by a methylene group (a:=1).

In some examples, a is 0 or 1, Y is 1,3-cyclopentyl, and b is 2 or 3. In other examples, a is 0 or 1, Y is 1,3-cyclohexyl or 1,4-cyclohexyl, and b is 1 or 2. Accordingly, some examples of the mitofusin activators disclosed herein having a=0 may have structures represented by Formulas 6-11 below,

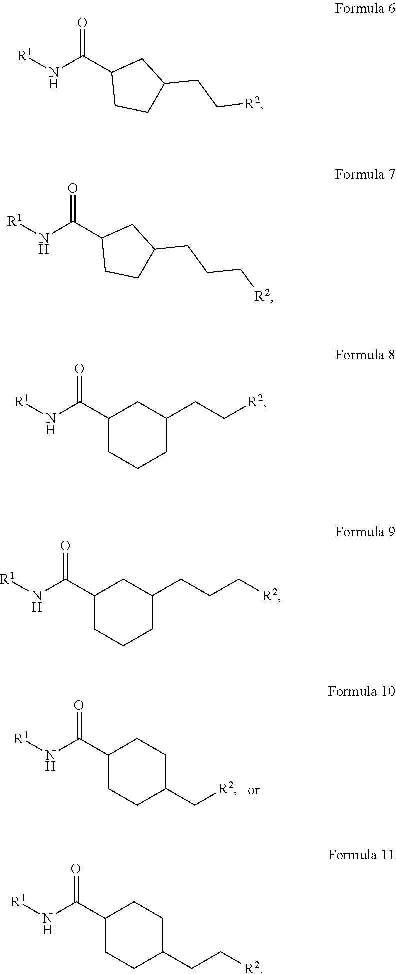

In some examples, a is 0 or 1, Y is 1,2-cyclopentyl or 1,2-cyclohexyl, and b is 2 or 3 Accordingly, some examples of the mitofusin activators disclosed herein having a=0 may have structures represented by Formulas 12-15 below.

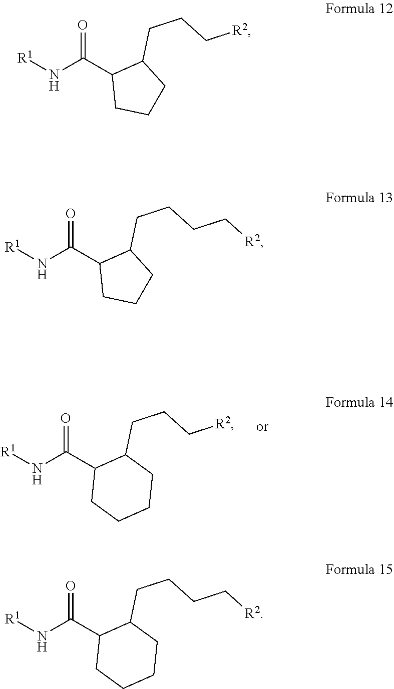

Still more specific examples of the mitofusin activators disclosed herein may have structures represented by Compounds 16-21 below

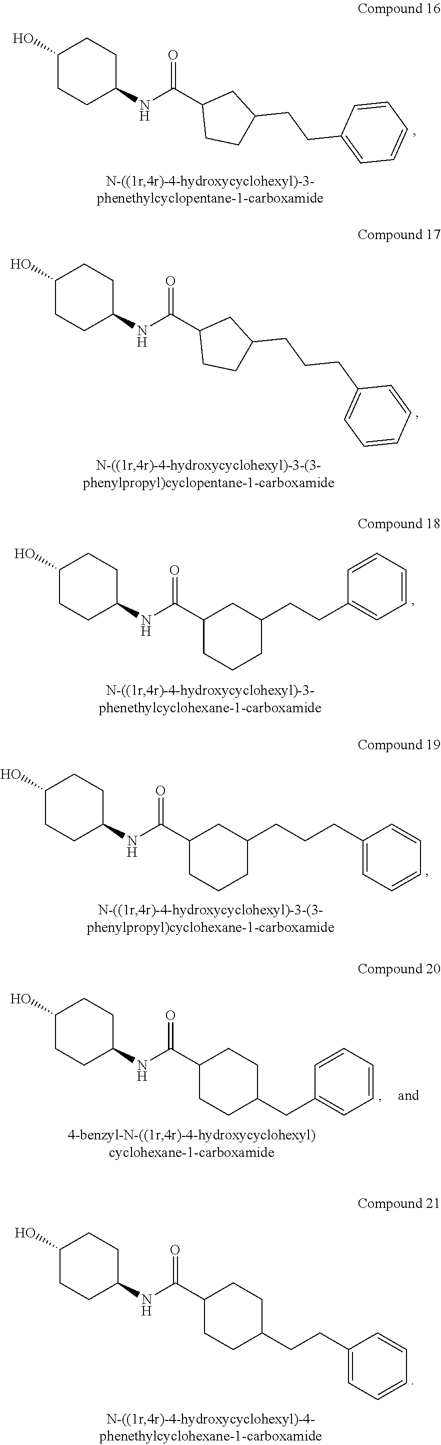 [SMILES: O=C(N[C@H]1CC[C@H](O)CC1)C1CCC(CCc2ccccc2)C1 ; O=C(N[C@H]1CC[C@H](O)CC1)C1CCC(CCCc2ccccc2)C1 ; O=C(N[C@H]1CC[C@H](O)CC1)C1CCCC(CCc2ccccc2)C1 ; O=C(N[C@H]1CC[C@H](O)CC1)C1CCCC(CCCc2ccccc2)C1 ; O=C(N[C@H]1CC[C@H](O)CC1)C1CCC(Cc2ccccc2)CC1 ; O=C(N[C@H]1CC[C@H](O)CC1)C1CCC(CCc2ccccc2)CC1]

### Embodiments Disclosed Herein Include

A. Compositions comprising a mitofusin activator. The mitofusin activator has a structure represented by

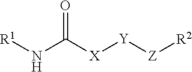

or a pharmaceutically acceptable salt thereof; wherein: R¹ is optionally substituted cycloalkyl or optionally substituted heterocycloalkyl; R² is optionally substituted phenyl; X is (CH₂)ₐ; Y is cyclopentyl or cyclohexyl, and Z is (CH₂)_{b}; wherein a is 0 or 1, and b is 1, 2 or 3.

B. Methods for administering a mitofusin activator to a subject having or suspected of having a mitochondria-associated disease. The methods comprise: administering a therapeutically effective amount of a composition comprising a mitofusin activator or a pharmaceutically acceptable salt thereof to a subject having or suspected of having a mitochondria-associated disease, disorder, or condition, the mitofusin activator having a structure represented by

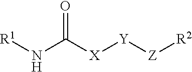

or a pharmaceutically acceptable salt thereof, wherein: R¹ is optionally substituted cycloalkyl or optionally substituted heterocycloalkyl; R² is optionally substituted phenyl; X is (CH₂)ₐ; Y is cyclopentyl or cyclohexyl; and Z is (CH₂)_{b}; wherein a is 0 or 1, and b is 1, 2 or 3.

Embodiments A and B may have one or more of the following additional elements in any combination.

Element 1: wherein X, Y and Z collectively form a 5- or 6-atom bridge between the amide carbonyl and R.

Element 2, wherein a is 0.

Element 3: wherein Y is 1,3-cyclopentyl, 1,3-cyclohexyl, or 1,4-cyclohexyl.

Element 4: wherein Y is 1,3-cyclopentyl, and b is 2 or 3.

Element 5: wherein Y is 1,3-cyclohexyl or 1,4-cyclohexyl, and b is 1 or 2.

Element 6: wherein the mitofusin activator has a structure represented by

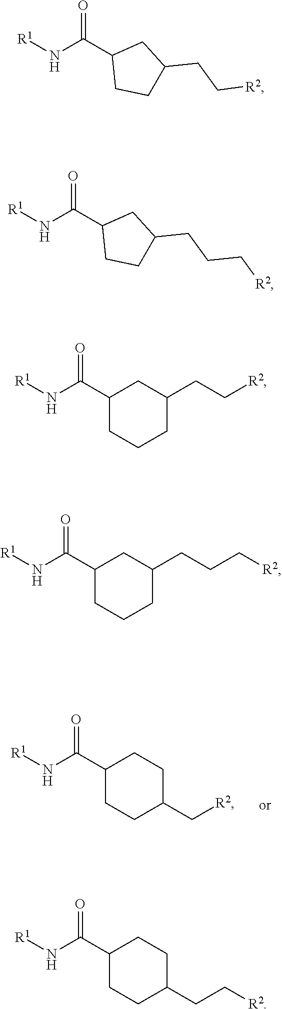

Element 7: wherein Y is 1,2-cyclopentyl or 1,2-cyclohexyl, and b is 2 or 3.

Element 8: wherein the mitofusin activator has a structure represented by

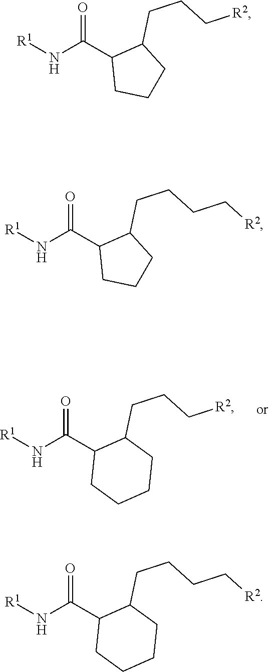

Element 9: wherein R¹ is trans-4-hydroxycyclohexyl.

Element 10: wherein the mitofusin activator has a structure selected from the group consisting of

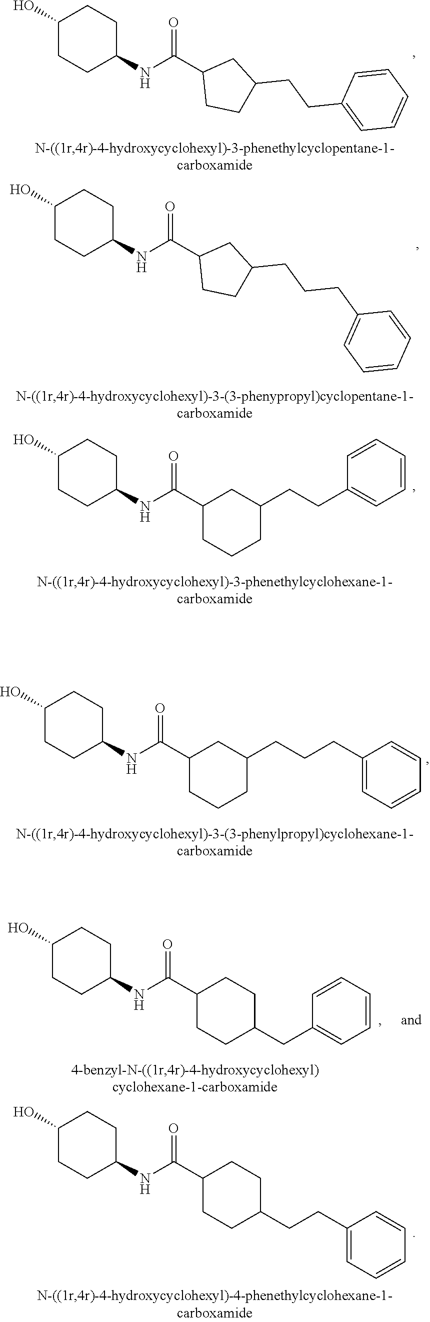 [SMILES: O=C(N[C@H]1CC[C@H](O)CC1)C1CCC(CCc2ccccc2)C1 ; O=C(N[C@H]1CC[C@H](O)CC1)C1CCC(CCCc2ccccc2)C1 ; O=C(N[C@H]1CC[C@H](O)CC1)C1CCCC(CCc2ccccc2)C1 ; O=C(N[C@H]1CC[C@H](O)CC1)C1CCCC(CCCc2ccccc2)C1 ; O=C(N[C@H]1CC[C@H](O)CC1)C1CCC(Cc2ccccc2)CC1 ; O=C(N[C@H]1CC[C@H](O)CC1)C1CCC(CCc2ccccc2)CC1]

Element 11: wherein the composition further comprises a pharmaceutically acceptable excipient.

Element 12: wherein the mitochondria-associated disease, disorder or condition is a peripheral nervous system (PNS) or central nervous system (CNS) genetic or non-genetic disorder, physical damage, and/or chemical injury.

Element 13: wherein the PNS or CNS disorder is one or more conditions selected from the group consisting of a chronic neurodegenerative condition in which mitochondrial fusion, fitness, and/or trafficking is/are impaired; a disease or disorder associated with mitofusin 1 (MFN1) or mitofusin 2 (MFN2) dysfunction; a disease associated with mitochondrial fragmentation, dysfunction, and/or dysmotility; a degenerative neuromuscular condition; Charcot-Marie-Tooth disease; Amyotrophic Lateral Sclerosis; Huntington's disease; Alzheimer's disease; Parkinson's disease; hereditary motor and sensory neuropathy; autism; autosomal dominant optic atrophy (ADOA); muscular dystrophy; Lou Gehrig's disease; cancer; mitochondrial myopathy; diabetes mellitus and deafness (DAD); Leber's hereditary optic neuropathy (LHON), Leigh syndrome; subacute sclerosing encephalopathy; neuropathy, ataxia, retinitis pigmentosa, and ptosis (NARP); myoneurogenic gastrointestinal encephalopathy (MNGIE); myoclonic epilepsy with ragged red fibers (MERRF), mitochondrial myopathy, encephalomyopathy, lactic acidosis, and stroke-like symptoms (MELAS); mtDNA depletion; mitochondrial neurogastrointestinal encephalomyopathy (MNGIE); dysautonomic mitochondrial myopathy; mitochondrial channelopathy; pyruvate dehydrogenase complex deficiency (PDCD/PDH); diabetic neuropathy; chemotherapy-induced peripheral neuropathy; crush injury; spinal cord injury (SCI); traumatic brain injury; stroke; optic nerve injury; conditions that involve axonal disconnection; and any combination thereof.

By way of non-limiting example, exemplary combinations applicable to A and B include, but are not limited to, 1 and 2; 1 and 3; 1 and 4; 1 and 5; and 6; 1 and 7; 1 and 8; 1 and 9; 1 and 10; 1 and 11; 1 and 12; 1 and 13; 2 and 3; 2 and 4; 2 and 5; 2 and 6; 2 and 7; 2 and 8; 2 and 9; 2 and 10; 2 and 11; 2 and 12; 2 and 13; 4 or 5, and 6; 2, 4 or 5, and 6; 4 or 5, and 9; 4 or 5, and 10; 4 or 5, and 11; 4 or 5, and 12; 4 or 5, and 13; 7 and 8; 7 and 9; and 11; 7 and 12; 7 and 13; 10 and 11; 10 and 12; and 10 and 13.

To facilitate a better understanding of the present disclosure, the following examples of preferred or representative embodiments are given. In no way should the following examples be read to limit, or to define, the scope of the invention.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

### EXAMPLES

### Methods

HPLC analyses were conducted with a Kinetex (C18 column (4.6×50 mm, 5 μm; Mobile phase A: 0.0375% TFA in water (v/v), B: 0.01875% TFA in Acetonitrile (v/v)) run at 50° C. with absorbance at 200 nm.

LC-MS/MS (ESI) was performed using 2 systems: 1) SHIMADZU LC-MS-2020 with LABSOLUTION V5.72 analysis software and a CHROMALITIH@FLASH RP-18E 25*2.0 mm column run at 50° C. with a PDA (220 and 254 nm) detector, acquired data in scan MS Mode (positive mode) with m/z=100-1000 scan range, drying gas (N₂) flow: 15 L/min, DL voltage: 120V and Quarry DC voltage: 20V, or 2) Agilent 1200/G6110A instrument with AgilentChemStation Rev. B. 04.03 software and an XBRIDGE C18 2.1*50 mm column run at 40° C. with DAD (220 nm)/ELSD detector, acquired data in scan MS Mode (positive mode) with m/z=100-1000 scan range, drying gas (N₂) flow: 10 L/min, 350° C., nebulizer pressure: 35 psi, capillary voltage: 2500V. NMR spectrometry was carried out on Brucker AVANCE NEO 400 MHz with a 5 mm PABBO BB/19F-1H/D Z-GRD probe.

Dose-response of mitofusin agonist fusogenicity was performed in Mfn1- or Mfn2-deficient MEFs (Mfn1-KO or Mfn2-KO MEFs) cultured at 37° C. and 5% CO₂-95% air. Cells were seeded on day I in 6 well plates at a density of 2×10⁴ cells per well and compounds added at 9 concentrations (0.5 nM-10 μM dissolved in DMSO) overnight. Mitochondria were then stained with MitoTracker Orange (200 nM; M7510; Invitrogen, Carlsbad, CA, USA). Nuclei were stained with Hoescht (10 gg/ml; Invitrogen, Thermo Fisher Scientific Cat: #1-3570). Images were acquired at room temperature on a Nikon Ti Confocal microscope using a 60×1.3 NA oil-immersion objective in Krebs-Henseleit buffer (138 NaCl, 3.7 nM KCl, 1.2 nM KH₂PO₄, 15 nM Glucose, 20 nM HEPES pH: 7.2-7.5, and 1 mM CaCl₂). Laser excitation was 549 nm with emission at 590 nm for MitoTracker Orange and excitation at 306 nm with emission at 405 nm for Hoescht. Images were analyzed using ImageJ and fusogenicity quantified as mitochondrial aspect ratio (length/width), and were indexed to the maximal response elicited by Compound 22, a known mitofusin activator. Response curves were interpolated using the sigmoidal model using Prism 8 software. EC₅₀ values are reported as mean with 95% confidence limits for at least 3 independent experiments.

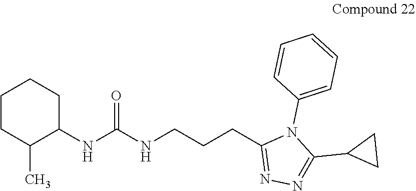 [SMILES: CC1CCCCC1NC(=O)NCCCc1nnc(C2CC2)n1-c1ccccc1]

### 1-(3-(5-cyclopropyl-4-phenyl-4H-1,2,4-triazol-3-yl)propyl)-3-(2-methylcyclohexyl)urea

In vitro pharmacokinetic analyses were performed in duplicate using standard methods by WuXi AppTec Co. Ltd. (Shanghai, China). Plasma protein binding was measured by equilibrium dialysis; % bound=(1−[free compound in dialysate]/[total compound in retentate])×100. Plasma stability of 2 uM compounds in clarified freeze-thawed plasma was assessed by LC-MS/MS of supernatants after protein precipitation: 120 min data are reported for studies including 0, 10, 30, 60, and 120 min. Liver microsome stability of 1 uM compounds in liver microsomes (0.5 mg/ml) after 0, 5, 10, 20, 30, 60 min. incubation was assessed by LC/MS/MS of reaction extracts.

Passive artificial blood brain barrier membrane permeability assay (PAMPA-BBB) were performed using 150 μL of 10 μM compounds (5% [DMSO) added to PVDF membranes pre-coated with 5 μL of 1% brain polar lipid extract (Porcine)/dodecane mixture and incubated for 4 h at room temperature with shaking at 300 rpm. Donor and acceptor samples were analyzed by LC-MS/MS.

### Synthesis

### Synthesis of N-(4-hydroxycyclohexyl)-3-phenethylcyclopentane-1-carboxamide (Compound 16)

Scheme 2 below outlines the synthesis of N-(4-hydroxycyclohexyl)-3-phenethylcyclopentane-1-carboxamide (Compound 16).

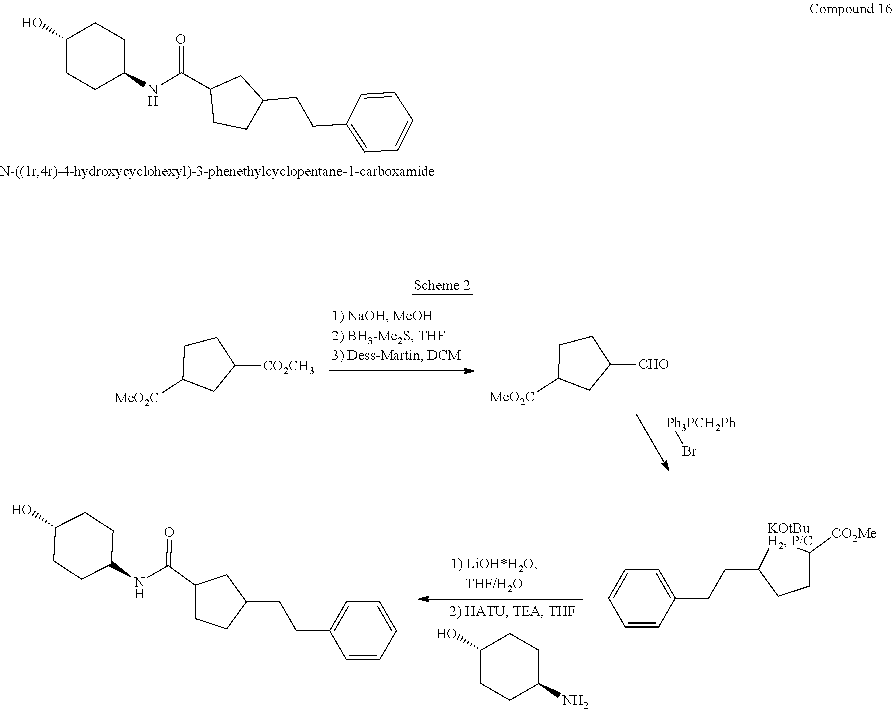

A mixture of dimethyl cyclopentane-1,3-dicarboxylate (2.00 g, 10.7 mmol, 1.00 eg) and NaOH (429 mg, 10.7 mmol, 1.00 eq) in MeOH (10.0 mL) was stirred for 12 hrs at 25° C., filtered and concentrated under reduced pressure to give a residue. The residue was purified by column chromatography to provide 3-(methoxycarbonyl)cyclopentane-1-carboxylic acid (1.20 g, 6.97 mmol, 64.8% yield) as a light yellow oil, To a mixture of 3-(methoxycarbonyl)cyclopentane-1-carboxylic acid (1.10 g, 6.39 mmol, 1.00 eq) in THF (10.0 mL) was added BH₃-Me₂S (10.0 M, 703 uL, 1.10 eq) at −78° C. The mixture was stirred for 0.5 hr, and then warmed to 25° C. and stirred for 12 hrs to obtain methyl 3-(hydroxymethyl)cyclopentane-1-carboxylate as a yellow oil (780 mg, 4.93 mmol, 77.1% yield). To a mixture of 3-(hydroxymethyl)cyclopentane-1-carboxylate (780 mg, 4.93 mmol, 1.00 eq) in DCM (10.0 mL) was added Dess-Martin periodinane (2.72 g, 6.41 mmol, 1.30 eq) at 25° C., and the mixture was stirred for 2 hrs, dried over Na₂SO₄, filtered and concentrated under reduced pressure to give a residue that was purified by column chromatography to obtain methyl 3-formylcycloheptane-1-carboxylate as a light yellow oil (283 mg, 1.81 mmol, 36.7% yield).

To a solution of benzyltriphenylphosphonium bromide (903 mug, 2.08 mmol, 1.15 eq) in THF (10.0 mL) was added t-BuOK (329 mg, 2.94 mmol, 1.62 eq) at −20° C. under N₂. The solution was stirred for 1 hr at −20° C. and then added to 3-formylcyclopentane-1-carboxylate (183 mg, 1.81 mmol, 1.00 eq) at −20° C. and stirred another 1 hr, filtered and concentrated under reduced pressure to give a residue that was purified by column chromatography to obtain methyl (E)-3-styrylcyclopentane-1-carboxylate (124 mg. 538 umol, 29.7% yield) as a light yellow oil. To a solution of (E)-3-styrylcyclopentane-1-carboxylate (124 mg, 538 umol, 1.00 eq) in MeOH (10.0 mL) was added Pd/C (120 mg, 10%) at 25° C. under H₂ (15 Psi. The mixture was stirred for 2 hrs at 25° C. to obtain methyl 3-phenethylcyclopentane-1-carboxylate (100 mg, 430 umol, 79.9% yield) as a light yellow oil.

To a mixture of 3-phenethylcyclopentane-1-carboxylate (85.0 tug, 365 umol, 1.00 eq) in MeOH (5.00 mL), THF (5.00 mL) and H₂O (2.00 mL) was added LiOH·H₂O (30.7 mug, 731 umol, 2.00 eq) at 25° C. The mixture was heated to 70° C. and stirred for 2 hrs to obtain 3-phenethylcyclopentane-1-carboxylic acid (79.0 mg, 361 umol, 98.9% yield) as a light yellow oil. To a mixture of 3-phenethylcyclopentane-1-carboxylic acid (79.0 ng, 361 umol, 1.00 eq) and (1r,4r)-4-aminocyclohexan-1-ol (65.8 mg, 434 umol, 1.20 eq) in THF (10.0 mL) was added HATU (206 mg, 542 umol, 1.50 eq) and Et₃N (727 mg, 7.18 mmol, 1.00 mL) at 25° C. and stirred for 12 hrs. The reaction mixture was concentrated under reduced pressure to give a residue. The residue was purified by preparative-HPLC (neutral condition, column: Waters Xbridge 150*25 mm*5 um; mobile phase: [water(NH₄HCO₃)-ACN]; B %: 43%-73%, 10 min). The title compound (8.24 mg, 25.2 umol, 6.97% yield, 96.5% purity) was obtained as a white solid. LCMS: RT=0.933 min, m/z 316.2 (M+H)⁺. HPLC: RT 2.130 mins, purity: 96.5% purity, under 220 nm. ¹H NMR: (400 MHz, DMSO-d₆) δ 7.52 (d, J=7.6, 1H), 7.27-7.23 (m, 2H), 7.18-7.15 (m, 3H), 4.52 (d, J=4.0, 1H), 3.43-3.41 (m, 1H), 2.57-2.53 (m, 2H), 1.87-1.57 (m, 12H), 1.18-1.06 (m, 71H).

### Synthesis of N-(4-hydroxycyclohexyl)-3-(3-phenylpropyl)cyclopentane-1-carboxamide (Compound 17)

Scheme 3 below outlines the synthesis of N-(4-hydroxycyclohexyl)-3-(3-phenylpropyl)cyclopentane-1-carboxamide (Compound 17).

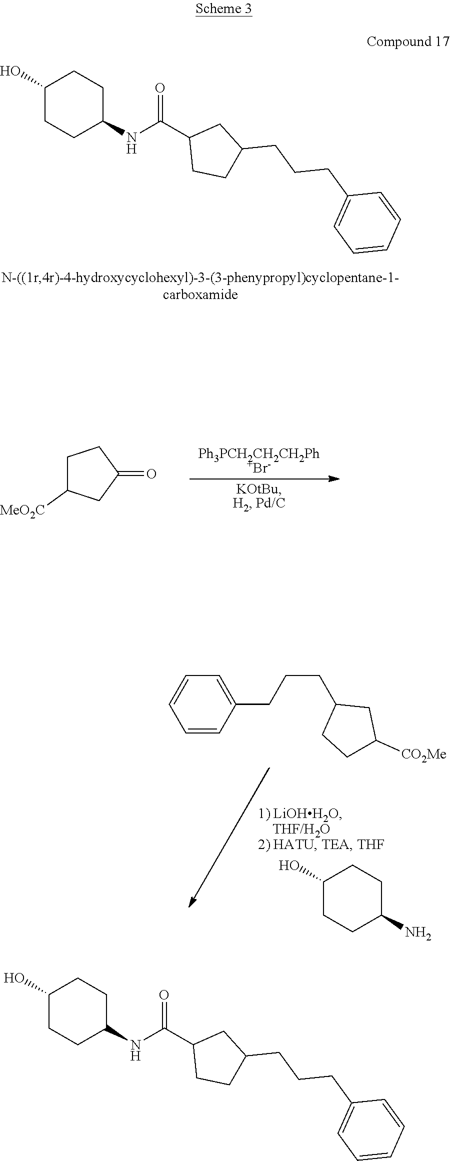

To a solution of triphenyl(3-phenylpropyl)phosphonium bromide (1.79 g, 3.87 mmol, 1.10 eq) in toluene (10.00 mL) was added t-BuOK (473 mg 4.22 mmol, 1.20 eq). The mixture was stirred at 70° C. for 1 hr, and methyl 3-oxocyclopentane-1-carboxylate (500 mg, 3.52 mmol, 1.00 eq) in toluene (2.00 mL) was added and stirred at 70° C. for 16 hrs to give methyl 3-43-phenylpropylidene)cyclopentane-1-carboxylate (0.100 g, 388 umol, 11.0% yield) as a colorless oil MeOH (10.0 mil) and Pd/C (0.10 g, 10%) were added to the 3-(3-phenylpropylidene)cyclopentane-1-carboxylate at 25° C. and degassed with N₂ for 3 times. The resulting mixture was stirred at 25° C. under H₂ (15 Psi) for 4 hrs. The mixture was filtered through Celite and the filtrate was concentrated under vacuum to provide methyl 3-(3-phenylpropyl)cyclopentane-1-carboxylate (90.0 mg, 365 umol, 94.1% yield) as a colorless oil.

A solution of 3-(3-phenylpropyl)cyclopentane-1-carboxylate (90.0 ng, 365 umol, 1.00 eq) and LiOH·H₂O (30.6 mg, 730 umol, 2.00 eq) in MeOH (5.00 mL) and H₂O (2.00 mL) was stirred at 70° C.; for 3 hrs to obtain 3-(3-phenylpropyl)cyclopentane-1-carboxylic acid (90.0 mg, crude) as a yellow oil. A mixture of 3-(3-phenylpropyl)cyclopentane-1-carboxylic acid (90.0 mg, 387 umol, 1.00 eq), (1r,4r)-4-aminocyclohexan-1-ol (49.0 mg, 426 umol, 1.10 eq), HATU (294 mg, 774 umol, 2.00 eq) and Et₃N (117 mg, 1.16 mmol, 161 μL, 3.00 eq) in THF (2.00 mL) was stirred at 25° C. for 16 hrs. The mixture was then concentrated to give a residue that was purified by preparative HPLC (Phenomenex Gemini-NX C18 75*30 mm*3 um; mobile phase: [water(0.225% FA)-ACN]; B %: 42%-72%, 7 min). The title compound (23.38 mg. 68.1 umol, 17.6% yield, 96.1% purity) was obtained as an off-white solid. LCMS: RT=0.859 min, m/z=330.3 (M+H)⁺. HPLC: RT=2.548 mins, purity: 96.1%, under 220 nm. ¹H NMR: (400 MHz, DMSO-d₆)δ 7.47 (d, J=8.0, 1H), 7.28-7.24 (m, 2H), 7.18-7.15 (m, 3H), 4.49 (d, J=4.0, 1H), 3.43-3.37 (m, 1H), 2.58-2.53 (m, 3H), 1.78-1.77 (m, 1H), 1.76-1.70 (m, 3H), 1.68-1.66 (m, 5H), 1.64-1.54 (m, 3H), 132-1.29 (m, 3H), 1.18-1.13 (m, 6H).

### Synthesis of N-(4-hydroxycyclohexyl)-3-phenethylcyclohexane-1-carboxamide (Compound 18)

Scheme 4 below outlines the synthesis of N-(4-hydroxycyclohexyl)-3-phenethylcyclohexane-1-carboxamide (Compound 18).

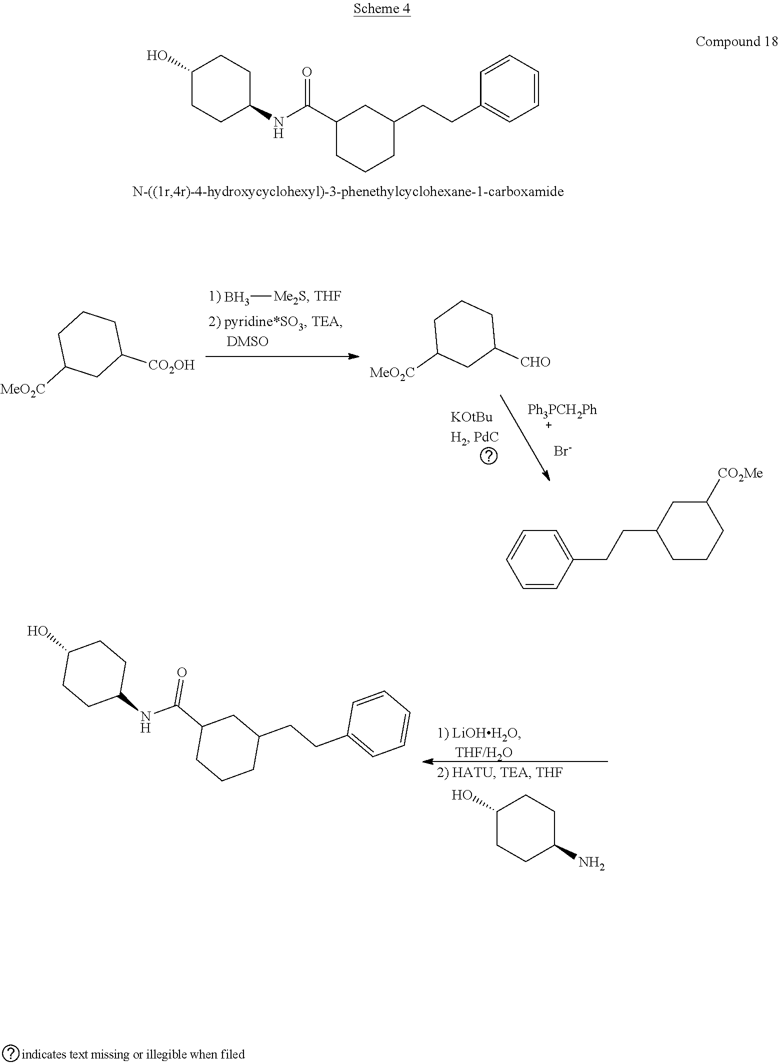

To a mixture of 3-(methoxycarbonyl)cyclohexane-1-carboxylic acid (2.00 g, 10.7 mmol, 1.00 eq in THY-11 (10.0 mL) was added BM₃-Me₂S (10.0 M, 1.18 mL, 1.10 eq) at −78° C., and the mixture was stirred for 0.5 hr at −78° C., warmed to 25° C. and stirred for 12 hrs to obtain methyl 3-(hydroxymethyl)cyclohexane-1-carboxylate (1.79 g, 10.4 mmol, 96.7% yield) as alight yellow oil. To a mixture of 3-(hydroxymethyl)cyclohexane-1-carboxylate (1.79 g, 10.4 mmol, 1.0.00 eq) and Et₃N (6.31 g, 62.3 mmol, 6.00 eq) in DMSO (20.0 mL) was added pyridine-sulfur trioxide (4.96 g, 31.2 mmol, 3.00 eq) at 25° C. and stirred for 12 hrs, dried over Na₂SO₄, filtered and concentrated under reduced pressure to give a residue. The residue was purified by column chromatography to obtain methyl 3-formylcyclohexane-1-carboxylate (710 mg, 4.17 mmol, 40.1% yield) as a yellow oil. To a solution of benzyltriphenylphosphonium bromide (1.17 g, 2.70 mmol, 1.15 eq) in THF (10.0 mL) was added t-BuOK (427 mg, 3.81 mmol, 1.62 eq) at −20° C. under N₂. The mixture was stirred for 1 hr at −20° C. and then 3-formylcyclohexane-1-carboxylate (400 mg, 2.35 mmol, 1.00 eq) was added at −20° C. and stirred another 1 hr. The reaction mixture was dried over Na₂SO₄, filtered and concentrated under reduced pressure to give a residue. The residue was purified by column chromatography to obtain methyl (E)-3-styrylcyclohexane-1-carboxylate (280 mg, 1.15 mmol, 48.7% yield) as a light yellow oil. The methyl (E)-3-styrylcyclohexane-1-carboxylate was then combined with MeOH (10.0 mL) and Pd/C (230 mg, 10%/o) at 25° C. under H₂ (15 Psi). The mixture was stirred for 2 hrs to obtain methyl 3-phenethylcyclohexane-1-carboxylate (230 mg, 933 umol, 87.7% yield) as a colorless oil.

To a mixture of 3-phenethylcyclohexane-1-carboxylate (165 mg, 669 umol, 1.00 eq) in MeOH (5.00 mL), THE (5.00 mL) and H₂O (2.00 mL) was added LiOH H₂O (56.2 mg, 1.34 umol, 2.00 eq) at 25° C., which was then heated to 70° C. and stirred for 2 hrs to obtain 3-phenethylcyclohexane-1-carboxylic acid (150 mg, 645 umol, 96.4% yield) as a light yellow oil. To a mixture of 3-phenethylcyclohexane-1-carboxylic acid (150 mg, 645 umol, 1.00 eq) and (1r,4r)-4-aminocyclohexan-1-ol (117 mg, 774 umol, 1.20 eq) in THF (10.0 mL) was added HATU (368 mg, 968 umol, 1.50 eq) and Et₃N (1.45 g, 14.3 mmol, 2.00 ml) at 25° C., stirred at 25° C. for 12 hrs. The reaction mixture was concentrated under reduced pressure to give a residue that was purified by preparative-HPLC (neutral condition, column: Waters Xbridge 150*25 mm*5 um; mobile phase: [water(NH₄HCO₃)-ACN]; B %: 40%-70%, 10 min). The title compound (8.15 mg, 24.3 umol, 3.78% yield, 98.6% purity) was obtained as a white solid. LCMS: RT 0.930 min, m/z=330.2 (M+H)⁺. HPLC: RT=2.218 mins, purity: 98.6%, under 220 nm. ¹H NMR: (400 MHz, DMSO-d₆)δ 7.46 (d, J=8.0, 1H), 7.27-7.24 (m, 2H), 7.19-7.14 (m, 31H), 4.49 (s, 1H), 3.42-3.38 (m, 1H), 2.59-2.55 (m, 211), 2.04-1.98 (m, 1H), 1.79-1.69 (m, 8H), 1.58-1.40 (m, 4H), 1.24-1.11 (m, 6H), 1.09-0.96 (m, 1H), 0.90-0.77 (m, 1H).

### Synthesis of 4-benzyl-N-(4-hydroxycyclohexyl)cyclohexane-1-carboxamide (Compound 20)

Scheme 5 below outlines the synthesis of 4-benzyl-N-(4-hydroxycyclohexyl)cyclohexane-1-carboxamide (Compound 20).

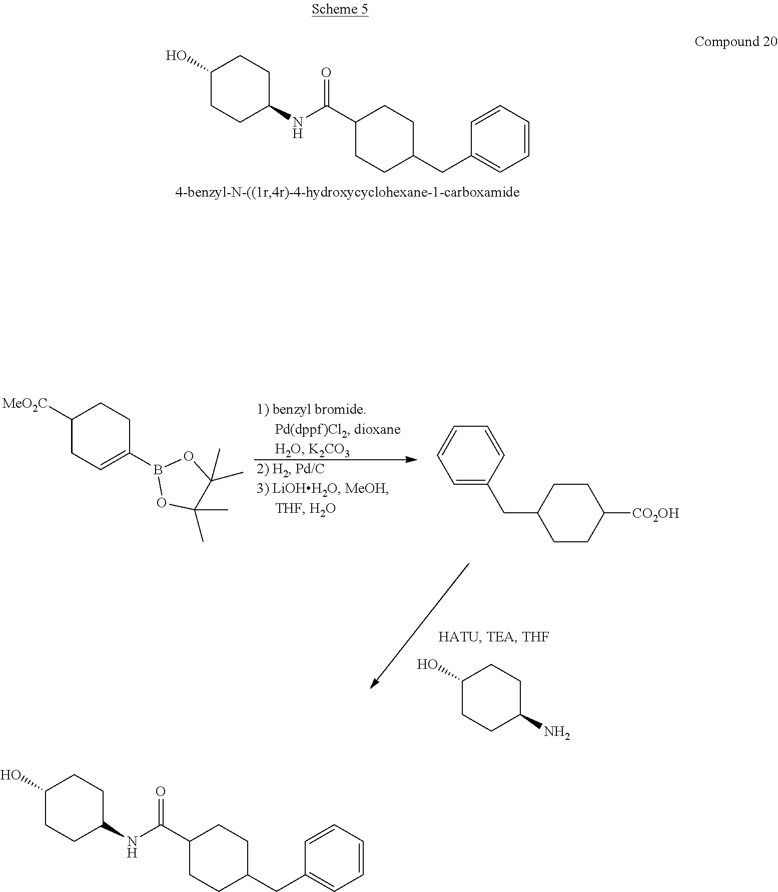

A mixture of methyl 4-(4,4,5,5-tetramethyl-1,3,2-dioxaborolan-2-yl)cyclohex-3-ene-1-carboxylate (500 mg. 1.88 mmol, 1.15 eq) and benzyl bromide (385 Ig, 2.25 mmol, 1.20 eq) in dioxane (10.0 mL) and H₂O (2.00 mL) was combined with Pd(dppf)Cl₂ (274 mg, 375 umol, 0.20 eq) and K₂CO₃ (778 mg, 5.64 mmol, 3.00 eq) at 25° C. under N₂. The mixture was heated to 100° C. and stirred for 2 hrs to obtain methyl 4-benzylcyclohex-3-ene-1-carboxylate (300 mg, 1.30 mmol, 69.3% yield) as a light yellow oil. To a solution of 4-benzylcyclohex-3-ene-1-carboxylate (300 mg, 1.30 mmol, 1.00 eq) in MeOH (10.0 mL) was added Pd/C (300 mg, 10%). The mixture was stirred at 25° C. under H₂ (15 Psi) for 2 hrs at 25° C. to obtain methyl 4-benzylcyclohexane-1-carboxylate (300 mg, 1.29 mmol, 99.1% yield) as a light yellow oil.

To a mixture of methyl 4-benzylcyclohexane-1-carboxylate (300 mg, 1.29 mmol, 1.00 eq) in MeOH (5.00 mL), THF (5.00 mL) and H₂O (2.00 mL) was added LiOH·H₂O (108 mg, 2.58 mmol, 2.00 eq) at 25° C., and the mixture was then heated to 70° C. and stirred for 2 hrs to obtain 4-benzylcyclohexane-1-carboxylic acid (280 ng, 1.28 mmol, 99.3% yield) as a light yellow oil. To a mixture of 4-benzylcyclohexane-1-carboxylic acid (280 mg, 1.28 mmol, 1.00 eq) and (1r,4r)-4-aminocyclohexan-1-ol (147 mg, 1.28 mmol, 1.00 eq) in THF (5.00 mL) was added HATU (731 mg, 1.92 mmol, 1.50 eg) and Et₃N (389 mg, 3.85 mmol, 3.00 eq) at 25° C. The mixture was stirred for 12 hrs. The reaction mixture was concentrated under reduced pressure to give a residue that was was purified by preparative-HPLC (neutral condition, column: Waters Xbridge 150*25 mm*5 um; mobile phase: [water(10 mM NH₄HCO₃)—ACN]; B %: 37%-67%, 10 min). The title compound (58.93 mg, 185 umol, 14.4% yield, 99.0% purity) was obtained as a white solid. LCMS: RT=0.823 min, m/z=316.3 (M+H)⁺. HPLC: RT=2.097 mins, purity: 99.0%, under 220 nm. ¹H NMR: (400 MHz, CDCl₃-d₁) δ 7.21-7.17 (m, 2H), 7.11-7.05 (m, 3H), 5.24 (d, J=7.6, 1H), 3.72-3.68 (m, 1H), 3.54-3.51 (m, 1H), 2.52 (d, J=7.6, 2H), 2.15-2.13 (m, 1H), 1.93-1.91 (m, 4H), 1.84-1.66 (m, 4H), 1.46-1.36 (m, 7H), 1.12-1.09 (m, 2H).

### Synthesis of N-(4-hydroxycyclohexyl)-4-phenethylcyclohexane-1-carboxamide (Compound 21)

Scheme 6 below outlines the synthesis of 4-benzyl-N-(4-hydroxycyclohexyl)cyclohexane-1-carboxamide (Compound 21).

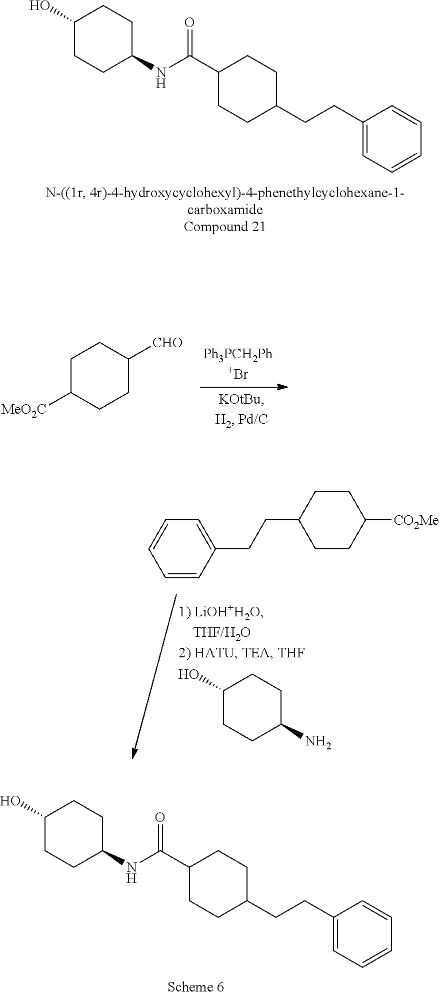 [SMILES: O=C(N[C@H]1CC[C@H](O)CC1)C1CCC(CCc2ccccc2)CC1 ; COC(=O)C1CCC(C=O)CC1 ; COC(=O)C1CCC(CCc2ccccc2)CC1 ; N[C@H]1CC[C@H](O)CC1 ; O=C(N[C@H]1CC[C@H](O)CC1)C1CCC(CCc2ccccc2)CC1]

A mixture of benzyltriphenylphosphonium bromide (1.47 g, 3.39 mmol, 1.15 eq) and t-BuOK (535 mg, 4.77 mmol, 1.62 eq) in THF (10.0 mL) at −20° C. under N₂ and was stirred for 1 hr, then was combined with 4-formylcyclohexane-1-carboxylic acid (500 mg, 2.94 mmol, 1.00 eq) at −20° C. and stirred another 1 hr. The reaction mixture was dried over Na₂SO₄, filtered and concentrated under reduced pressure to give a residue that was purified by column chromatography to obtain methyl (E)-4-styrylcyclohexane-1-carboxylate (130 mg, 530 umol, 18.1% yield, 99.7% purity) as an off-white solid. The (E)-4-styrylcyclohexane-1-carboxylate (130 mg, 530 umol, 18.1% yield, 99.7% purity) was then added to MeOH (10.0 mL) and Pd/C (120 mg, 10%) at 25° C. and stirred under H₂ (15 Psi) stirred for 2 hrs to obtain methyl 4-phenethylcyclohexane-1-carboxylate (120 mg, 487 umol, 91.5% yield) as a light yellow oil.

To a mixture of methyl 4-phenethylcyclohexane-1-carboxylate (120 mg. 487 umol, 1.00 eq) in MeOH (5.00 mL), THF (5.00 mL) and H₂O (2.00 mL) was added LiOH·H₂O (40.9 mg, 974 umol, 2.00 eq) at 25° C., which was then heated to 70° (C and stirred for 2 hrs to obtain 4-phenethylcyclohexane-1-carboxylic acid (110 mg, 473 umol, 97.2% yield) as a light yellow oil. The 4-phenethylcyclohexane-1-carboxylic acid (110 mg. 473 umol, 97.2% yield) was then combined with (1r,4r)-4-aminocyclohexan-1-ol (65.4 mg, 568 umol, 1.20 eq) in THF (10.0 mL), HATU (270 mg, 710 umol, 1.50 eq) and Et₃N (143 mg, 1.42 mmol, 3.00 eq) at 25° C. The reaction mixture was stirred for 12 hrs, concentrated under reduced pressure, and purified by preparative HPLC (neutral condition, column: Waters Xbridge 150*25 mm*5 um; mobile phase: [water(10 mM NH₄HCO₃)-ACN]; B %: 40%-70%, 10 min). The title compound 21 (9.82 mg, 29.3 umol, 6.21% yield, 98.6% purity) was obtained as a white solid. LCMS: RT 0.859 min, m/z=330.3 (M+I) HPLC: RT=2.236 mins, purity: 98.6%, under 220 nm. ¹H NMR: (400 MHz, DMSO-d₆)δ 7.45 (d, J=8.0, 1H), 7.27-7.24 (m 2H), 7.18-7.15 (m, 3H), 4.48 (d, 4.0, 1H), 3.46-3.36 (m, 2H), 2.59-2.55 (m, 2H), 2.03-1.96 (m, 1H), 1.80-1.76 (m, 4H), 1.69-1.64 (m, 4H), 1.45-1.43 (m, 2H), 1.35-1.25 (m, 2H), 1.25-1.13 (m, 5H), 0.91-0.87 (m, 2H).

Scheme 7 below outlines the synthesis of 4-benzyl-N-(4-hydroxycyclohexyl)cyclohexane-1-carboxamide (Compound 21).

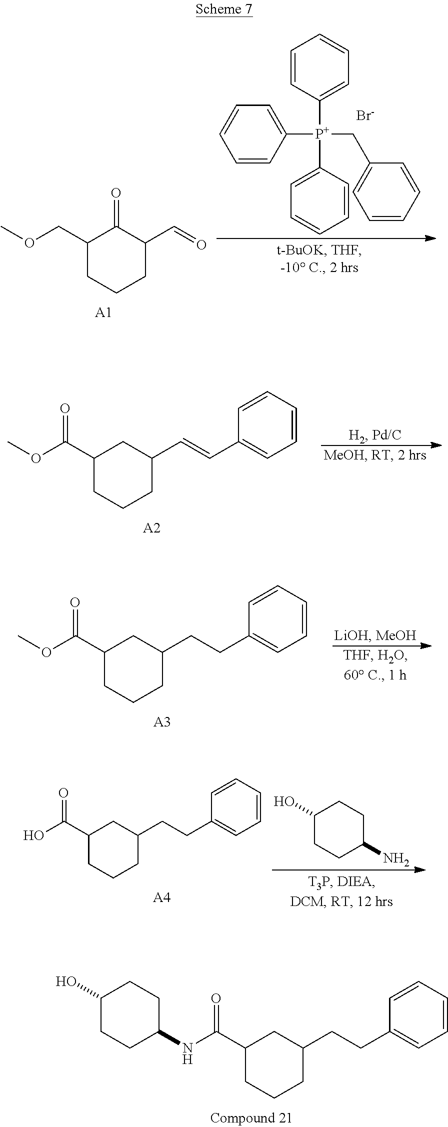

### Preparation of Compound A2

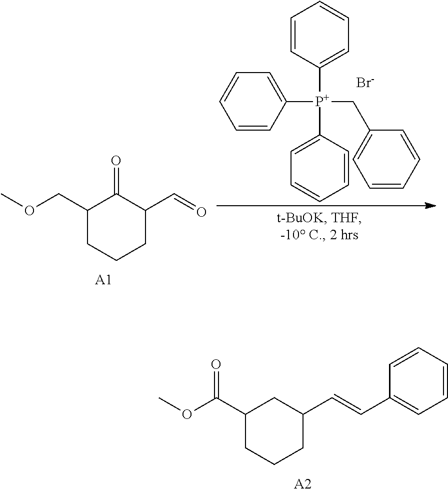

To a solution of benzyl(triphenyl)phosphonium; bromide (380 mg, 876.95 umol, 1.0 eq) in THF (2 mL) was added t-BuOK (196.81 mg, 1.75 mmol, 2.0 eg) at −10° C. The mixture was stirred at −10° C. for 1 hr. Then a solution of methyl 3-formylcyclohexanecarboxylate (179.11 mg, 1.05 mmol, 1.2 eq) in THF (0.5 mL) was added to the mixture at −10° C. The mixture was stirred at −10° C. for 1 hr. The reaction mixture was quenched by addition sat. aq. NH₄Cl 5 mL at 0° C., and extracted with EtOAc (5 mL*3). The combined organic layers were dried over Na₂SO₄, filtered and concentrated under reduced pressure to give a residue. The residue was purified by column chromatography (silica gel, 100-200 mesh, 0-100% ethyl acetate in petroleum ether) affording (E)-methyl 3-styrylcyclohexanecarboxylate (100 mg, 409.29 umol, 46.67% yield) as a colorless oil. ESI [M+H]=245.2.

### Preparation of Compound A3

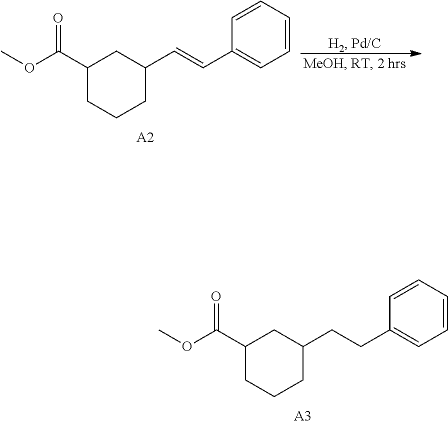

A mixture of (E)-methyl 3-strylcyclohexanecaraxylate (100 mg, 409.29 umol, 1.0 eq) and Pd/C (20 mg, 10% purity) in MeOH (2 mL) was degassed and purged with H₂ for 3 times, then the mixture was stirred at 25° C. for 2 hrs under H₂ atmosphere (15 psi). The reaction mixture was concentrated to give methyl 3phenethylycyclohexanecarboxylate (50 mg, crude) as a colorless oil. ESI [M+H]=247.2.

### Preparation of Compound A4

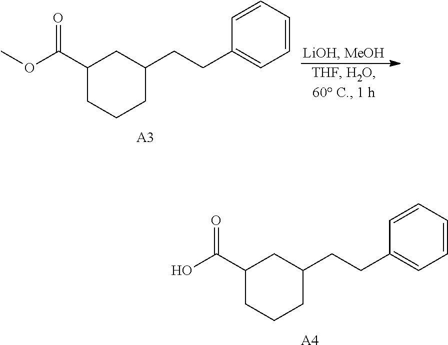

A mixture of methyl 3-phenethylcyclohexanecarboxylate (50 mg, 202.97 umol, 1.0 eq) and LiOH·-H₂O (17.03 mg. 405.94 umol, 2.0 eq) in MeOH (1 mL), THF (1 mL) and H₂O (0.5 mL) was stirred at 60° C. for 2 hrs. The reaction mixture was concentrated tinder reduced pressure to give a residue. The residue was added water (1 mL) and extracted with DCM (1 mL*2). The aqueous was adjusted to pH=3 with 3M HCl at 0° C., and extracted with DCM (5 mL*3). The combined organic layers were dried over Na₂SO₄, filtered and concentrated to give 3-phenethylcyclohexanecarboxylic acid (40 mg, crude) as a yellow oil. ESI [M+H]=233.2.

### Preparation of Compound 21

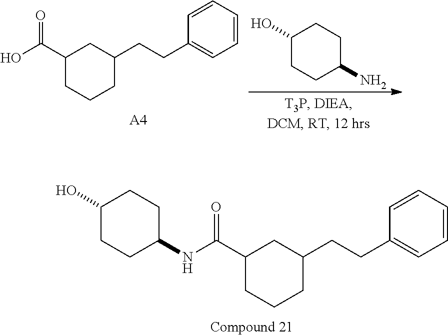

To a solution of 3-phenethylcyclohexanecarbaxylic acid (40 mg, 172.18 umol, 1.0 eq), 4-aminocyclohexanol (59.49 mg. 516.53 umol, 3 eq) and DIEA (66.76 mg, 516.53 umol, 3.0 eq) in DCM (2 mL) was added T₃P (219.14 mg, 344.36 umol, 50% purity, 2.0 eq) (in EtOAc). The mixture was stirred at 25° C. for 3 hr. The reaction mixture was concentrated and the residue was purified by prep-HPLC (column: Waters Xbridge BEH C18 100*30 mm*10 um; mobile phase: [water(—NH₄HCO₃)-ACN]; B %: 45%-75%, 8 min) to give N-((1r,4r)-4-hydroxycyclohexyl)-3-phenethylcyclohexanecarboxamide (10.0 ng, 30.35 umol, 17.63% yield, 100% purity) as a white solid. ¹H NMR (400 MHz, MeOD-d4) δ 7.29-7.24 (m, 2H), 7.23-7.14 (n, 31) 3.66-3.50 (m, 2H), 2.69-2.61 (m, 21), 2.20-2.11 (m, 111), 1.98 (br d, J=11.7 Hz, 2H), 1.93-1.79 (m, 5H), 1.78-1.65 (m, 1H), 1.61-1.53 (m, 2H), 1.45-1.23 (m, 7H), 1.17 (q, J=12.2 Hz, 1H), 1.03-0.88 (m, 1H). ESI [M+H]=330.2.

Mitofusin Activity and In Vitro Pharmacology. Table 1 below summarizes the biological activity of Compounds 1, 2, 4, and 23-27 (Comparative Examples) in comparison to Compounds 16, 17, 18, 20, and 21 (Experimental Examples).

TABLE 1

| | | Plasma Protein | | Liver Microsomes | | PAMPA |
|---|---|---|---|---|---|---|
| | EC₅₀ | Binding (Bound %) | | (t_{1/2}, minutes) | | (10⁻⁶ |
| Compound | (nm) | Human | Mouse | Human | Mouse | cm/s) |
| 1 (Comp.) | 5.8 | 91 | 96.3 | >145 | 92.4 | 26 |
| 2 (Comp.) | 6.1 | 94.4 | 95.5 | >145 | 114.1 | 58 |
| 4 (Comp.) | 11 | 97.2 | 94.3 | >145 | 105.5 | 61 |
| 14 (Exp.) | 7 | 97.7 | 96.0 | >145 | 35.1 | 114 |
| 15 (Exp.) | 15.3 | 99.3 | 98.8 | 120.2 | 43.2 | 154 |
| 16 (Exp.) | 4.8 | 97.8 | 4.0 | 69.2 | 20.5 | 126 |
| 18 (Exp.) | 44.6 | 92.1 | 94.4 | >145 | 49.3 | 88.8 |
| 19 (Exp.) | 7.4 | 99.6 | 98.9 | >145 | 74.4 | 133 |
| 21 (Comp.) | 19.4 | — | — | 53.6 | 9.9 | 240 |
| 22 (Comp.) | 2.7 | — | — | >145 | 137.5 | 7.1 |
| 23 (Comp.) | 6.3 | — | — | 23.7 | 111.9 | 9.6 |
| 24 (Comp.) | >5 | — | — | 7 | 1.4 | 86.7 |
| 25 (Comp.) | 46.9 | 98.8 | 96.1 | 10.2 | 2.8 | 215 |

As shown, Compound 2 and Compound 1 exhibited similar EC₅₀ values for mitofusin activation, while the PAMPA value of Compound 2 was over twice the value of Compound 1. The higher PAMPA value is characteristic of greater passive blood-brain barrier permeability. The EC₅₀ value and elevated PAMPA value remained elevated in the compound having Compound 4.

It was found that increasing the cycloalkyl ring size from cyclopropyl (Compound 2) to cyclopentyl or cyclohexyl (Compounds 16-21) increased the PAMPA values, while still maintaining low EC₅₀ values in most cases. In contrast, there was not an appreciable PAMPA increase upon increasing the ring size from cyclopropyl (Compound 2) to cyclobutyl (Compound 4).

### EQUIVALENTS

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

The foregoing description has been presented only for the purposes of illustration and is not intended to limit the disclosure to the precise form disclosed, but by the claims appended hereto.

1. A compound having a structure represented by Formula (I) 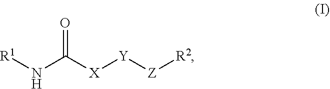
or a pharmaceutically acceptable salt thereof, wherein:
R¹ is C₃-C₁₀ cycloalkyl or 3- to 10-membered heterocycloalkyl, wherein the cycloalkyl or heterocycloalkyl is optionally substituted with one or more R^{X};
each R^{X} independently is halogen, cyano, —OR^{X1}, —N(R^{X1})₂, oxo, C₁-C₁₀ alkyl, or C₃-C₁₀ cycloalkyl;
R² is phenyl optionally substituted with one or more R^{Y};
each R^{Y} independently is halogen, cyano, —OR^{Y1}, —N(R^{Y1})₂, C₁-C₁₀ alkyl, or C₃-C₁₀ cycloalkyl;
each R^{X1} and R^{Y1} independently is H or C₁-C₆ alkyl;
X is (CH₂)ₐ;
Y is cyclopentyl or cyclohexyl;
Z is (CH₂)_{b};
a is 0 or 1; and
b is 1, 2, or 3.

2. The compound of claim 1, wherein X, Y, and Z collectively form a 5- or 6-atom bridge between the amide carbonyl and R².

3. The compound of claim 1, wherein a is 0.

4. The compound of claim 3, wherein Y is 1,3-cyclopentyl, 1,3-cyclohexyl, or 1,4-cyclohexyl.

5. The compound of claim 3, wherein Y is 1,3-cyclopentyl, and b is 2 or 3.

6. The compound of claim 3, wherein Y is 1,3-cyclohexyl or 1,4-cyclohexyl, and b is 1, 2, or 3.

7. The compound of claim 3, wherein the compound has a structure represented by 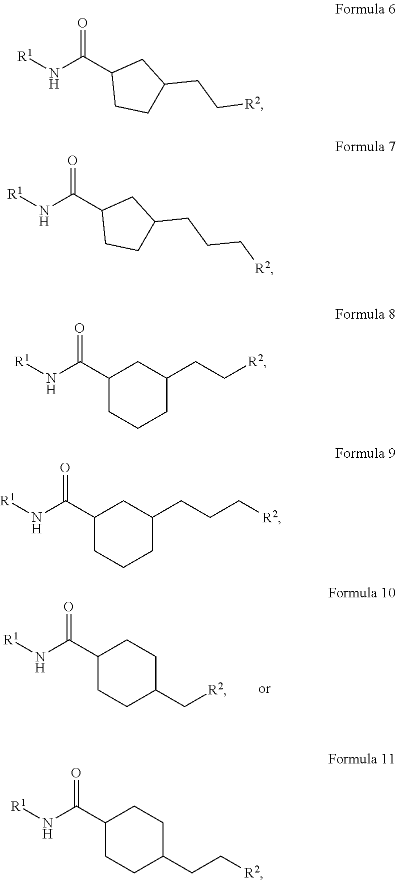
or a pharmaceutically acceptable salt thereof.

8. The compound of claim 3, wherein the compound has a structure represented by 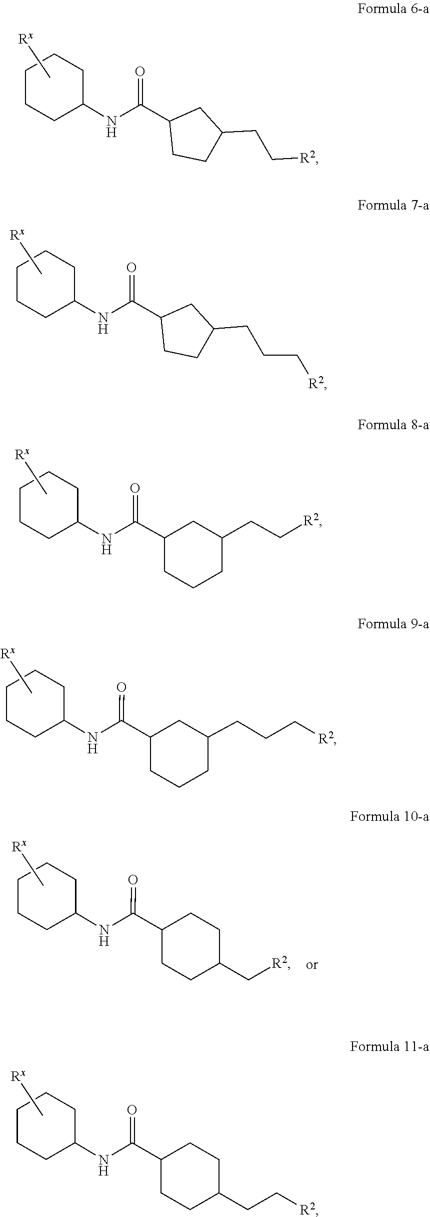
or a pharmaceutically acceptable salt thereof.

9. The compound of claim 3, wherein the compound has a structure represented by 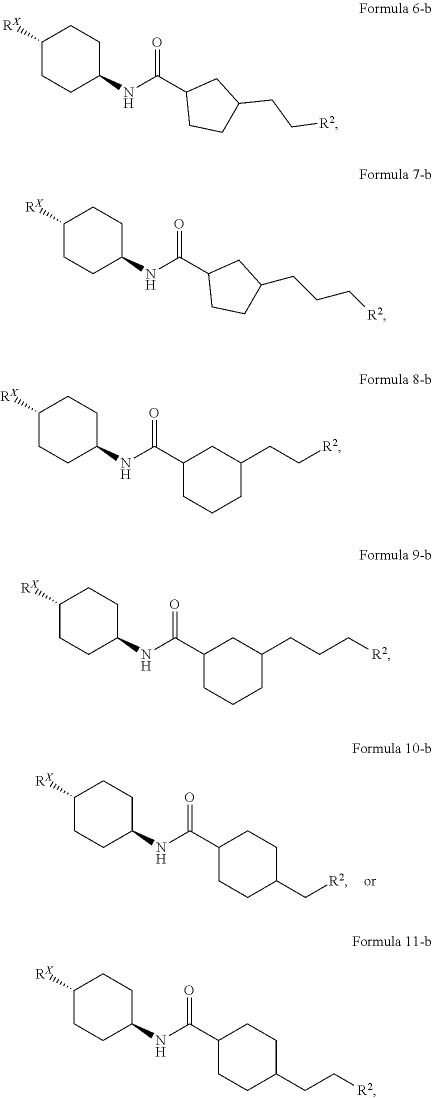
or a pharmaceutically acceptable salt thereof.

10. The compound of claim 3, wherein the compound has a structure represented by 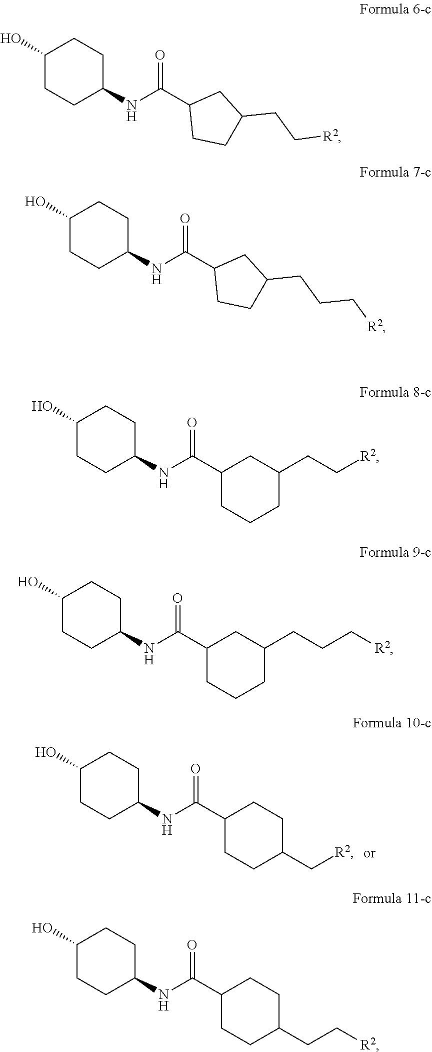
or a pharmaceutically acceptable salt thereof.

11. The compound of claim 3, wherein the compound has a structure represented by 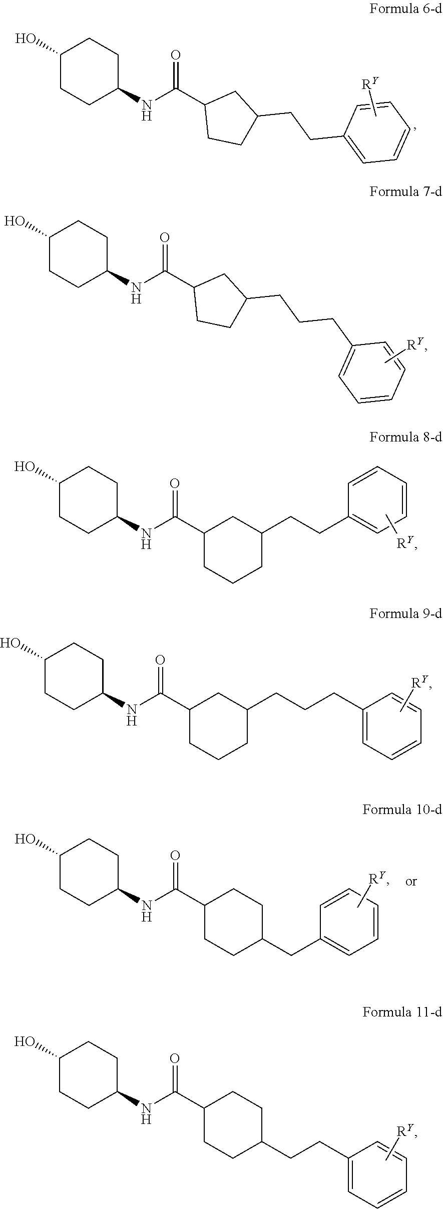
or a pharmaceutically acceptable salt thereof.

12. (canceled)

13. (canceled)

14. (canceled)

15. (canceled)

16. (canceled)

17. (canceled)

18. The compound of claim 1, wherein R¹ is trans-4-hydroxycyclohexyl.

19. The compound of claim 18, wherein the compound has a structure selected from the group consisting of 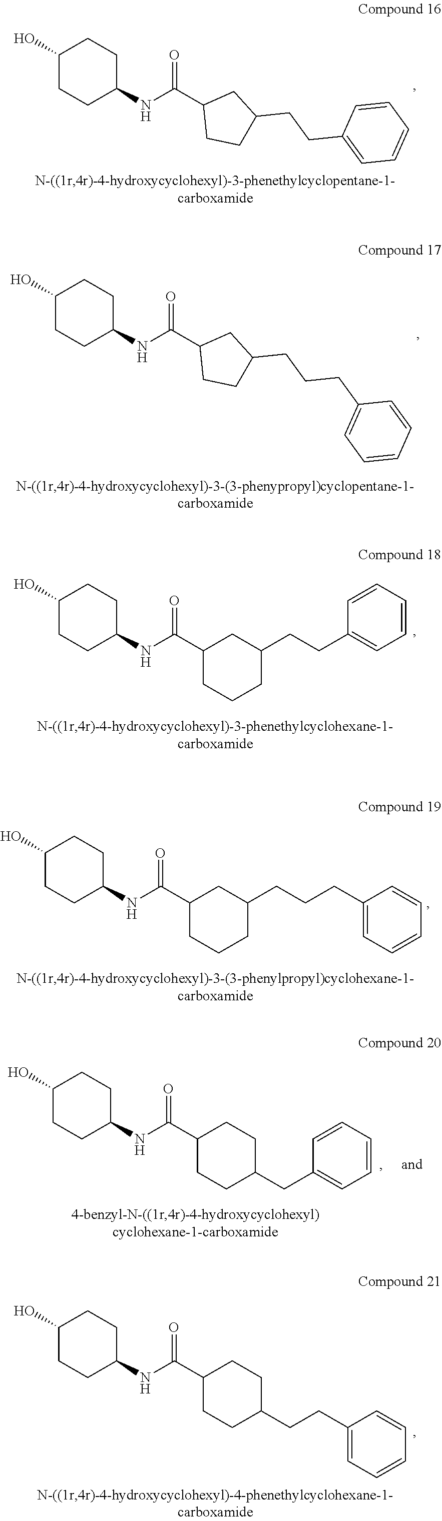 [SMILES: O=C(N[C@H]1CC[C@H](O)CC1)C1CCC(CCc2ccccc2)C1 ; O=C(N[C@H]1CC[C@H](O)CC1)C1CCC(CCCc2ccccc2)C1 ; O=C(N[C@H]1CC[C@H](O)CC1)C1CCCC(CCc2ccccc2)C1 ; O=C(N[C@H]1CC[C@H](O)CC1)C1CCCC(CCCc2ccccc2)C1 ; O=C(N[C@H]1CC[C@H](O)CC1)C1CCC(Cc2ccccc2)CC1 ; O=C(N[C@H]1CC[C@H](O)CC1)C1CCC(CCc2ccccc2)CC1]
or a pharmaceutically acceptable salt thereof.

20. A pharmaceutical composition, comprising the compound of claim 1 and a pharmaceutically acceptable excipient.

21. A method of treating or preventing a disease, disorder, or condition in a subject, comprising administering the compound of claim 1 or a pharmaceutical composition comprising the compound of claim 1 and a pharmaceutically acceptable excipient to the subject.

22. (canceled)

23. (canceled)

24. (canceled)

25. The method of claim 21, wherein the disease, disorder, or condition is associated with mitochondria or responsive to mitofusin modulation.

26. (canceled)

27. The method of claim 21, wherein the disease, disorder, or condition is peripheral nervous system (PNS) or central nervous system (CNS) genetic or non-genetic disorder, physical damage, or chemical injury.

28. The method of claim 27, wherein the PNS or CNS genetic or non-genetic disorder is one or more conditions selected from the group consisting of a chronic neurodegenerative condition in which mitochondrial fusion, fitness, and/or trafficking is/are impaired; a disease or disorder associated with mitofusin 1 (MFN1) or mitofusin 2 (MFN2) dysfunction; a disease associated with mitochondrial fragmentation, dysfunction, and/or dysmotility; a degenerative neuromuscular condition; Charcot-Marie-Tooth disease; Amyotrophic Lateral Sclerosis; Huntington's disease; Alzheimer's disease; Parkinson's disease; hereditary motor and sensory neuropathy; autism; autosomal dominant optic atrophy (ADOA); muscular dystrophy; Lou Gehrig's disease; cancer; mitochondrial myopathy; diabetes mellitus and deafness (DAD); Leber's hereditary optic neuropathy (LHON); Leigh syndrome; subacute sclerosing encephalopathy; neuropathy, ataxia, retinitis pigmentosa, and ptosis (NARP); myoneurogenic gastrointestinal encephalopathy (MNGIE); myoclonic epilepsy with ragged red fibers (MERRF); mitochondrial myopathy, encephalomyopathy, lactic acidosis, and stroke-like symptoms (MELAS); mtDNA depletion; mitochondrial neurogastrointestinal encephalomyopathy (MNGIE); dysautonomic mitochondrial myopathy; mitochondrial channelopathy; pyruvate dehydrogenase complex deficiency (PDCD/PDH); diabetic neuropathy; chemotherapy-induced peripheral neuropathy; crush injury; spinal cord injury (SCI); traumatic brain injury; stroke; optic nerve injury; conditions that involve axonal disconnection; and any combination thereof.

29. A method of activating mitofusin in a subject, comprising administering the compound of claim 1 or a pharmaceutical composition comprising the compound of claim 1 and a pharmaceutically acceptable excipient to the subject.

30. (canceled)

31. (canceled)

32. (canceled)
